(19) United States
(12) Patent Application Publication
(10) Pub. No.: US 2024/0108245 A1
(43) Pub. Date: Apr. 4, 2024

(54) ANOMALY DETECTION DEVICE, DETERMINATION SYSTEM, ANOMALY DETECTION METHOD, AND PROGRAM RECORDING MEDIUM

(71) Applicant: NEC Corporation, Tokyo (JP)

(72) Inventors: Chenhui HUANG, Tokyo (JP); Kenichiro FUKUSHI, Tokyo (JP)

(73) Assignee: NEC Corporation, Tokyo (JP)

(21) Appl. No.: 18/538,109

(22) Filed: Dec. 13, 2023

Related U.S. Application Data
(63) Continuation of application No. 17/790,228, filed on Jun. 30, 2022.

(51) Int. Cl. A61B 5/11; A61B 5/00

(52) U.S. Cl. CPC A61B 5/11; A61B 5/6807

(57) ABSTRACT

An anomaly detection device that includes an extraction unit that acquires sensor data from a sensor installed in footwear and extract a gait feature amount characteristic in gait of a pedestrian wearing the footwear by using the sensor data, and a detection unit that detects an anomaly in a foot of the pedestrian walking wearing the footwear based on the gait feature amount extracted by the extraction unit.

The present application is a continuation application of U.S. patent application Ser. No. 17/790,228 filed on Jun. 30, 2022, which is a National Stage Entry of international application PCT/JP2020/000698 filed on Jan. 10, 2020, the contents of all of which are incorporated herein by reference, in their entirety.

### TECHNICAL FIELD

The present invention relates to an anomaly detection device and the like that detect an anomaly in a foot of a pedestrian.

### BACKGROUND ART

With an increase in interest in healthcare that manages physical condition, a service for measuring a gait including a gait feature of a pedestrian and providing information according to the gait to a user has attracted attention. For example, hallux valgus is one of foot anomalies caused by gait features. Since hallux valgus gradually progresses, it is sometimes unnoticed until it grows incurable. Hallux valgus is mainly caused by compatibility between the footwear and the foot, and the gait has a feature. Therefore, if the risk of hallux valgus can be detected based on the features of gait, it may be possible to suppress the progress of hallux valgus.

PTL 1 discloses a foot part analyzer that performs analysis of shapes of a foot and a toe. The device of PTL 1 includes sensors for measuring force acting on predetermined positions of a component with which the sole of a foot comes into contact and a sensor for measuring whether a scaphoid bone has moved. The device of PTL 1 determines whether pronation has occurred based on output from the sensors to determine the existence of anomaly in the foot.

### CITATION LIST

### Patent Literature

[PTL 1] JP 2019-150229 A

### SUMMARY OF INVENTION

### Technical Problem

By using the device of PTL 1, anomaly in the foot can be detected by measuring the pressure applied by the site relevant to the navicular bone of the sole. However, measurement of the pressure has had a problem of being susceptible to body motion noise. Since the installation of the sensor for measuring the foot pressure is fixed, there has been a problem that the sensor cannot be applied to various foot shapes.

An object of the present invention is to provide an anomaly detection device and the like capable of detecting an anomaly in a foot based on features of gait of a pedestrian.

### Solution to Problem

An anomaly detection device of one aspect of the present invention includes: an extraction unit that acquires sensor data from a sensor installed in footwear and extract a gait feature amount characteristic in gait of a pedestrian wearing the footwear by using the sensor data; and a detection unit that detects an anomaly in a foot of the pedestrian walking wearing the footwear based on the gait feature amount extracted by the extraction unit.

In an anomaly detection method of one aspect of the present invention, a computer acquires sensor data from a sensor installed in footwear, extracts a gait feature amount characteristic in gait of a pedestrian wearing the footwear by using the sensor data, and detects an anomaly in a foot of the pedestrian walking wearing the footwear based on the extracted gait feature amount.

A program of one aspect of the present invention causes a computer to execute processing of acquiring sensor data from a sensor installed in footwear, processing of extracting a gait feature amount characteristic in gait of a pedestrian wearing the footwear by using the sensor data, and processing of detecting an anomaly in a foot of the pedestrian walking wearing the footwear based on the extracted gait feature amount.

### Advantageous Effects of Invention

According to the present invention, it is possible to provide an anomaly detection device and the like capable of detecting an anomaly in a foot based on features of gait of a pedestrian.

### BRIEF DESCRIPTION OF DRAWINGS

FIG. 1 is a block diagram illustrating an example of a configuration of a determination system according to a first example embodiment.

FIG. 2 is a conceptual diagram illustrating an example in which a data acquisition device of the determination system according to the first example embodiment is installed in footwear.

FIG. 3 is a conceptual diagram for explaining a relationship between a local coordinate system of the data acquisition device of the determination system and a world coordinate system according to the first example embodiment.

FIG. 4 is a conceptual diagram for explaining a plantar angle calculated by an anomaly detection device of the determination system according to the first example embodiment.

FIG. 5 is a conceptual diagram for explaining hallux valgus.

FIG. 6 is a conceptual diagram for explaining a general gait cycle.

FIG. 7 is a conceptual diagram for explaining gait waveform data extracted by a detection unit of the determination system according to the first example embodiment.

FIG. 8 is a block diagram illustrating an example of a configuration of the data acquisition device of the determination system according to the first example embodiment.

FIG. 9 is a block diagram illustrating an example of a configuration of the anomaly detection device of the determination system according to the first example embodiment.

FIG. 10 is a conceptual diagram for explaining an example in which the anomaly detection device of the determination system according to the first example embodiment estimates a progression state of hallux valgus by using a first model.

FIG. 11 is a conceptual diagram for explaining an example in which the anomaly detection device of the determination system according to the first example embodiment estimates a hallux valgus (HV) angle by using a second model.

FIG. 12 is a conceptual diagram for explaining an example in which the anomaly detection device of the determination system according to the first example embodiment distributes content according to the progression state of hallux valgus.

FIG. 13 is a conceptual diagram for explaining another example in which the anomaly detection device of the determination system according to the first example embodiment distributes content according to the progression state of hallux valgus.

FIG. 14 is a conceptual diagram for explaining a photographing condition of a camera for measuring the HV angle of a subject.

FIG. 15 is a conceptual diagram for explaining an example in which positions of a first metatarsal bone and a first proximal phalanx are extracted from an image photographed for measuring the HV angle of the subject.

FIG. 16 is a conceptual diagram for explaining a feature site extracted from gait waveform data of an angular velocity (roll angular velocity) about an X axis obtained by gait of the subject wearing footwear in which the data acquisition device of the determination system according to the first example embodiment is disposed.

FIG. 17 is a graph obtained by plotting, with respect to the gait speed, components having a gait cycle of 73% of the roll angular velocity obtained by gait of the subject wearing footwear in which the data acquisition device of the determination system according to the first example embodiment is disposed.

FIG. 18 is a graph obtained by plotting, with respect to the gait speed, distances between a regression line and components having a gait cycle of 73% of the roll angular velocity obtained by gait of the subject wearing footwear in which the data acquisition device of the determination system according to the first example embodiment is disposed.

FIG. 19 is a box-and-whisker diagram illustrating variation in distance between the regression line and the components having a gait cycle of 73% of the roll angular velocity obtained by gait of the subject wearing footwear in which the data acquisition device of the determination system according to the first example embodiment is disposed.

FIG. 20 is a conceptual diagram for explaining a feature site extracted from gait waveform data of acceleration in a gravity direction (Z direction acceleration) obtained by gait of the subject wearing footwear in which the data acquisition device of the determination system according to the first example embodiment is disposed.

FIG. 21 is a graph obtained by plotting, with respect to the gait speed, components having a gait cycle of 73% of the Z direction acceleration obtained by gait of the subject wearing footwear in which the data acquisition device of the determination system according to the first example embodiment is disposed.

FIG. 22 is a graph obtained by plotting, with respect to the gait speed, distances between a regression line and components having a gait cycle of 73% of the Z direction acceleration obtained by gait of the subject wearing footwear in which the data acquisition device of the determination system according to the first example embodiment is disposed.

FIG. 23 is a box-and-whisker diagram illustrating variation in distance between the regression line and the components having a gait cycle of 73% of the Z direction acceleration obtained by gait of the subject wearing footwear in which the data acquisition device of the determination system according to the first example embodiment is disposed.

FIG. 24 is a conceptual diagram for explaining a feature site extracted from gait waveform data of acceleration in a traveling direction (Y direction acceleration) obtained by gait of the subject wearing footwear in which the data acquisition device of the determination system according to the first example embodiment is disposed.

FIG. 25 is a graph obtained by plotting, with respect to the gait speed, components having a gait cycle of 43% of the Y direction acceleration obtained by gait of the subject wearing footwear in which the data acquisition device of the determination system according to the first example embodiment is disposed.

FIG. 26 is a graph obtained by plotting, with respect to the gait speed, distances between a regression line and components having a gait cycle of 43% of the Y direction acceleration obtained by gait of the subject wearing footwear in which the data acquisition device of the determination system according to the first example embodiment is disposed.

FIG. 27 is a box-and-whisker diagram illustrating variation in distance between the regression line and the components having a gait cycle of 43% of the Y direction acceleration obtained by gait of the subject wearing footwear in which the data acquisition device of the determination system according to the first example embodiment is disposed.

FIG. 28 is a graph obtained by plotting, with respect to the gait speed, components having a gait cycle of 73% of the Y direction acceleration obtained by gait of the subject wearing footwear in which the data acquisition device of the determination system according to the first example embodiment is disposed.

FIG. 29 is a graph obtained by plotting, with respect to the gait speed, distances between a regression line and components having a gait cycle of 73% of the Y direction acceleration obtained by gait of the subject wearing footwear in which the data acquisition device of the determination system according to the first example embodiment is disposed.

FIG. 30 is a box-and-whisker diagram illustrating variation in distance between the regression line and the components having a gait cycle of 73% of the Y direction acceleration obtained by gait of the subject wearing footwear in which the data acquisition device of the determination system according to the first example embodiment is disposed.

FIG. 31 is a flowchart for explaining an example of an operation of an extraction unit of the anomaly detection device of the determination system according to the first example embodiment.

FIG. 32 is a flowchart for explaining an example of the operation of the detection unit of the anomaly detection device of the determination system according to the first example embodiment.

FIG. 33 is a flowchart for explaining an example of a selection method of a gait feature amount extracted by the extraction unit of the anomaly detection device of the determination system according to the first example embodiment.

FIG. 34 is a flowchart for explaining an example of the selection method of a gait feature amount extracted by the extraction unit of the anomaly detection device of the determination system according to the first example embodiment.

FIG. 35 is a block diagram illustrating an example of a configuration of an anomaly detection device according to a second example embodiment.

FIG. 36 is a block diagram for explaining an example of a hardware configuration that implements the anomaly detection device according to each example embodiment.

### EXAMPLE EMBODIMENT

Example embodiments of the present invention will be described below with reference to the drawings. The example embodiments described below have technically desirable limitations for carrying out the present invention, but the scope of the invention is not limited to the following. In all the drawings used in the description of the example embodiments below, the same reference signs are given to similar parts unless there is a particular reason. In the following example embodiments, repeated description regarding similar configurations and operations may be omitted.

### First Example Embodiment

First, the determination system according to the first example embodiment will be described with reference to the drawings. The determination system of the present example embodiment determines the presence or absence of an anomaly in a foot of a pedestrian using sensor data acquired by a sensor installed in footwear. In particular, the determination system of the present example embodiment determines whether the foot of a pedestrian has a risk of hallux valgus using the sensor data acquired by an inertial measurement device installed under an arch of foot of the pedestrian.

(Configuration)

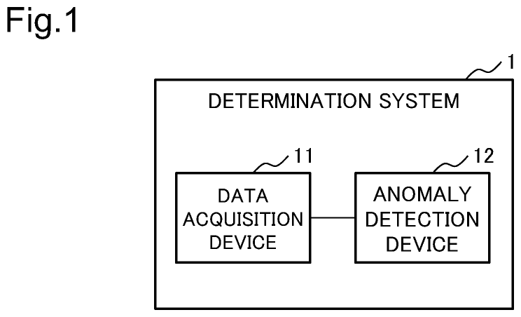

FIG. 1 is a block diagram illustrating an example of the configuration of a determination system 1 of the present example embodiment. As in FIG. 1, the determination system 1 includes a data acquisition device 11 and an anomaly detection device 12. The data acquisition device 11 and the anomaly detection device 12 may be connected by wire or may be connected wirelessly. The data acquisition device 11 and the anomaly detection device 12 may be configured by a single device. Alternatively, the data acquisition device 11 may be excluded from the configuration of the determination system 1, and only the anomaly detection device 12 may constitute the determination system 1.

The data acquisition device 11 includes a sensor installed in footwear. The data acquisition device 11 converts a physical quantity acquired by the sensor into digital data (also referred to as sensor data), and transmits the converted sensor data to the anomaly detection device 12.

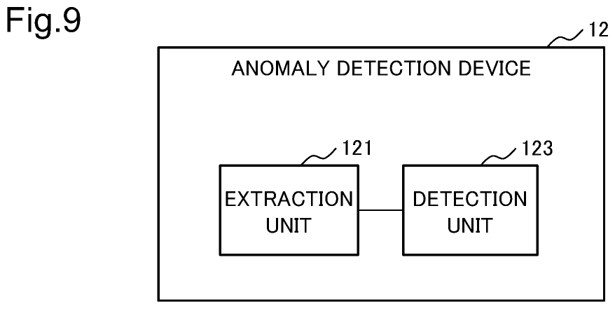

As illustrated in FIG. 9, the anomaly detection device 12 includes an extraction unit 121 and a detection unit 123. The extraction unit 121 acquires sensor data from the sensor installed in the footwear. The anomaly detection device 12 extracts a gait feature amount characteristic in gait of the pedestrian wearing the footwear using the acquired sensor data. The detection unit 123 detects an anomaly in the foot of the pedestrian walking wearing the footwear based on the gait feature amount extracted by the extraction unit 121. The anomaly detection device 12 can detect an anomaly in the foot based on a feature of gait of the pedestrian.

The determination system 1 of the present example embodiment can be applied to determination of a progression state of hallux valgus. Next, an example of the configuration of the determination system 1 capable of determining the progression state of hallux valgus will be described in detail.

The sensor used in the data acquisition device 11 includes at least an acceleration sensor and an angular velocity sensor. For example, the data acquisition device 11 is installed in an insole to be inserted into footwear. In a case of determining the progression state of hallux valgus, the data acquisition device 11 is desirably installed at a position below the arch of foot. The data acquisition device 11 converts physical quantities such as acceleration and angular velocity acquired by the acceleration sensor and the angular velocity sensor into digital data (also referred to as sensor data), and transmits the converted sensor data to the anomaly detection device 12.

The data acquisition device 11 is implemented by, for example, an inertial measurement device including an acceleration sensor and an angular velocity sensor. An example of the inertial measurement device is an inertial measurement unit (IMU). The IMU includes a three-axis acceleration sensor and a three-axis angular velocity sensor. Furthermore, examples of the inertial measurement device include a vertical gyro (VG), an attitude heading (AHRS), and a global positioning system/inertial navigation system (GPS/INS).

Sensor data such as acceleration and angular velocity acquired by the data acquisition device 11 are also referred to as gait parameters. The speed and angle calculated by integrating acceleration and angular velocity are also included in the gait parameters. In the present example embodiment, a lateral direction of the pedestrian is an X direction (right side is positive), a traveling direction of the pedestrian is a Y direction (front side is positive), and a gravity direction is a Z direction (upper side is positive). In the present example embodiment, rotation about the X axis is defined as roll, rotation about the Y axis is defined as pitch, and rotation about the Z axis is defined as yaw.

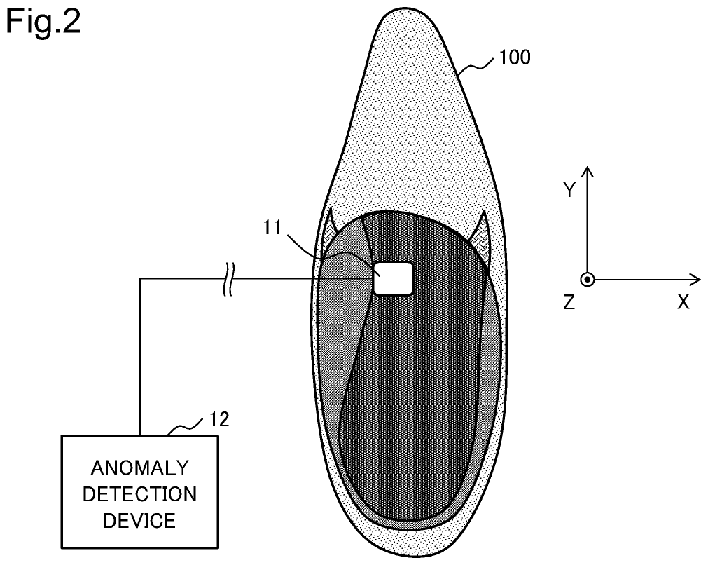

FIG. 2 is a conceptual diagram illustrating an example in which the data acquisition device 11 is installed in a shoe 100. In the example of FIG. 2, the data acquisition device 11 is installed at a position relevant to the back side of the arch of foot. For example, the data acquisition device 11 is installed in an insole to be inserted into the shoe 100. Note that the data acquisition device 11 may be installed at a position other than the back side of the arch of foot as long as the risk of progressing to hallux valgus can be detected.

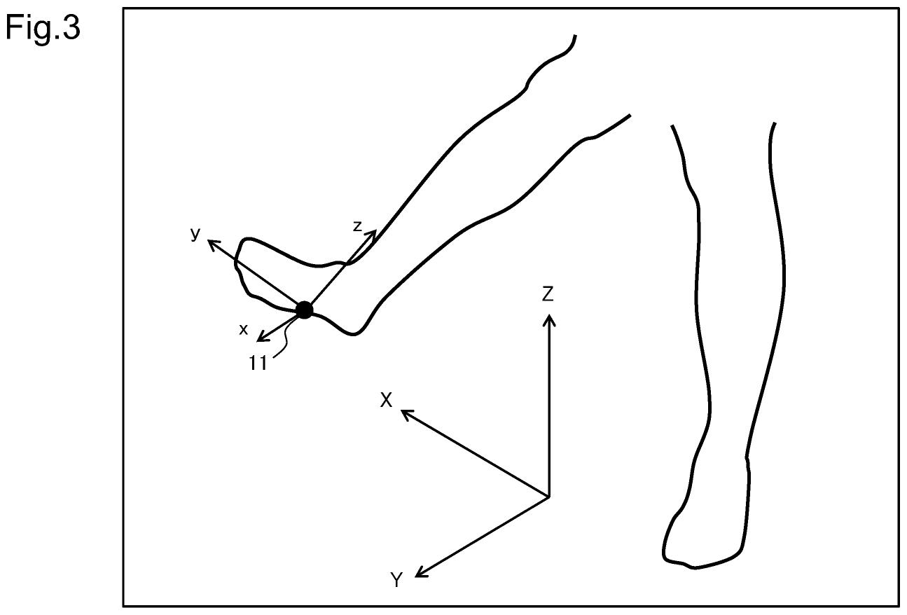

FIG. 3 is a conceptual diagram for explaining a local coordinate system (x axis, y axis, z axis) set in the data acquisition device 11 and a world coordinate system (X axis, Y axis, Z axis) set with respect to the ground in a case where the data acquisition device 11 is installed on the back side of the arch of foot. In the world coordinate system (X axis, Y axis, Z axis), in a state where a pedestrian is standing upright, the lateral direction of the pedestrian is set to an X axis direction (rightward direction is positive), the front direction (traveling direction) of the pedestrian is set to a Y axis direction (forward direction is positive), and the gravity direction is set to a Z axis direction (vertically upward direction is positive). In a state where the pedestrian is standing upright, the local coordinate system (x axis, y axis, z axis) and the world coordinate system (X axis, Y axis, Z axis) are consistent. According to the walking of pedestrians, the spatial posture of the data acquisition device 11 changes, and therefore the local coordinate system (x axis, y axis, z axis) and the world coordinate system (X axis, Y axis, Z axis) are inconsistent. Therefore, the anomaly detection device 12 converts the sensor data acquired by the data acquisition device 11 from the local coordinate system (x axis, y axis, z axis) of the data acquisition device 11 into the world coordinate system (X axis, Y axis, Z axis).

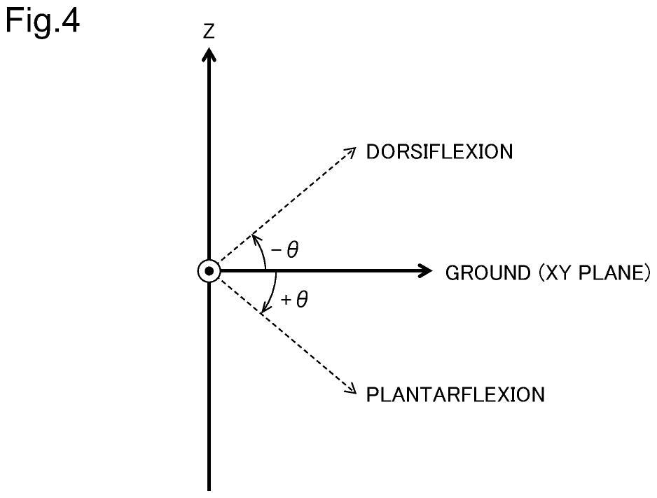

For example, the anomaly detection device 12 calculates a plantar angle. FIG. 4 is a conceptual diagram for explaining the plantar angle calculated by the anomaly detection device 12. The plantar angle is the angle of the bottom of the foot with respect to the ground (XY plane). The plantar angle is defined as minus in a state where the toe faces upward (dorsiflexion), and as plus in a state where the toe faces downward (plantarflexion).

For example, the anomaly detection device 12 calculates the plantar angle using the magnitude of the acceleration in each axial direction of the X axis and the Y axis. For example, the anomaly detection device 12 can calculate the plantar angle about each of the X axis, the Y axis, and the Z axis by integrating the values of the angular velocity having each of the X axis, the Y axis, and the Z axis as the central axis. Acceleration data and angular velocity data include high-frequency noise and low-frequency noise that change in various directions. Therefore, by applying a low-pass filter and a high-pass filter to the acceleration data and the angular velocity data to remove a high-frequency component and a low-frequency component, it is possible to improve accuracy of sensor data from a foot on which noise is easily included. By applying a complementary filter to each of the acceleration data and the angular velocity data to take a weighted mean, it is possible to improve accuracy of sensor data.

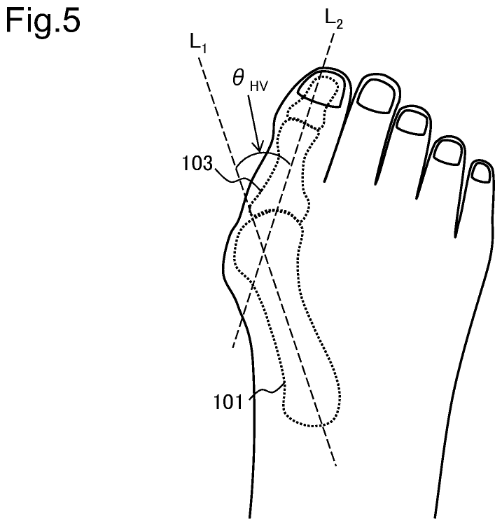

FIG. 5 is a conceptual diagram for explaining hallux valgus. In FIG. 5, a first metatarsal bone 101 and a first proximal phalanx 103 are indicated by dotted lines. Hallux valgus is a symptom in which the thumb of the foot turns to be valgus, and hallux valgus is accompanied by varus of the first metatarsal bone 101. If a pedestrian continues to walk wearing footwear that does not fit the foot, a force is applied in a direction where the first metatarsal bone 101 is varus, which increases the risk of progression of hallux valgus. The progression state of hallux valgus is determined by an angle (HV angle θ_{HV}) formed by a center line L₁ of the first metatarsal bone 101 and a center line L₂ of the first proximal phalanx 103 (HV: Hallux valgus). The state in which the HV angle θ_{HV} exceeds 20 degrees is hallux valgus. Hallux valgus is affected not only by the compatibility between the footwear and the foot but also by the feature of gait. The feature of gait of a person with hallux valgus will be described later.

The anomaly detection device 12 acquires sensor data in the local coordinate system from the data acquisition device 11. The anomaly detection device 12 converts the acquired sensor data in the local coordinate system into the world coordinate system to generate time series data. The anomaly detection device 12 extracts gait waveform data for one gait cycle from the generated time series data. The anomaly detection device 12 extracts a feature site regarding an anomaly in the foot from the extracted gait waveform data for one gait cycle. In particular, the anomaly detection device 12 extracts a feature site regarding hallux valgus from the extracted gait waveform data for one gait cycle.

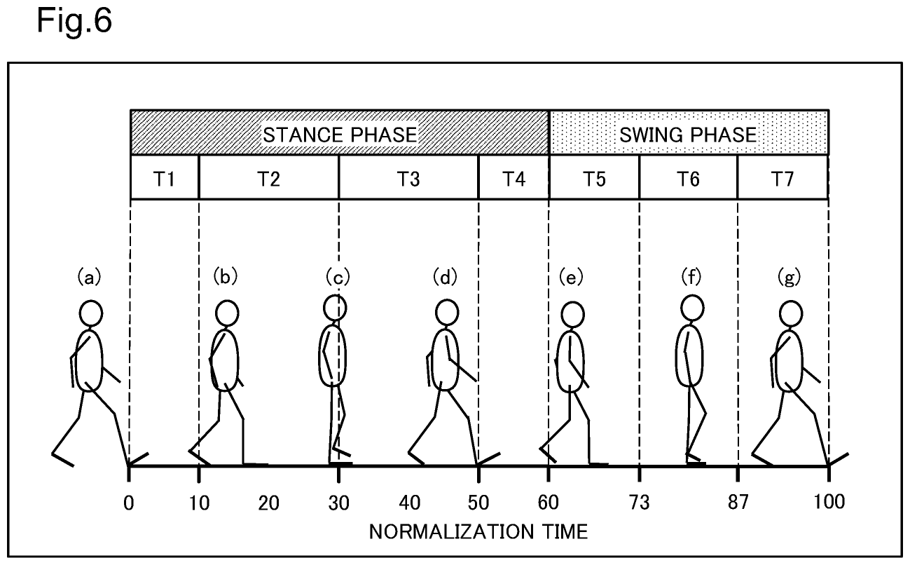

FIG. 6 is a conceptual diagram for explaining a general gait cycle. FIG. 6 illustrates one gait cycle of a right foot. The horizontal axis in FIG. 6 represents time (also referred to as normalization time) normalized with one gait cycle of the right foot as 100%, where a time point at which the heel of the right foot lands on the ground as a start point and a time point at which the heel of the right foot next lands on the ground as an end point. In general, one gait cycle of one foot is roughly divided into a stance phase in which at least a part of the back side of the foot is in contact with the ground and a swing phase in which the back side of the foot is away from the ground. The stance phase is subdivided into an initial stance period T1, a mid-stance period T2, a terminal stance period T3, and a pre-swing period T4. The swing phase is further subdivided into an initial swing period T5, a mid-swing period T6, and a terminal swing period T7.

In FIG. 6, (a) expresses a situation in which the heel of the right foot comes into contact with the ground (heel contact). (a) is a start point of one gait cycle. (b) expresses a situation in which the toe of the left foot is separated from the ground in a state where the entire sole of the right foot is in contact with the ground (contralateral toe off). (c) expresses a situation in which the heel of the right foot is lifted in a state where the entire sole of the right foot is in contact with the ground (heel lift). (d) is a situation in which the heel of the left foot is in contact with the ground (contralateral heel contact). (e) expresses a situation in which the toe of the right foot is separated from the ground in a state where the entire sole of the left foot is in contact with the ground (toe off). (f) expresses a situation in which the left foot and the right foot cross each other in a state where the entire sole of the left foot is in contact with the ground (foot crossing). (g) expresses a situation in which the heel of the right foot comes into contact with the ground (heel contact). (g) is an end point of one gait cycle and the start point of a next gait cycle.

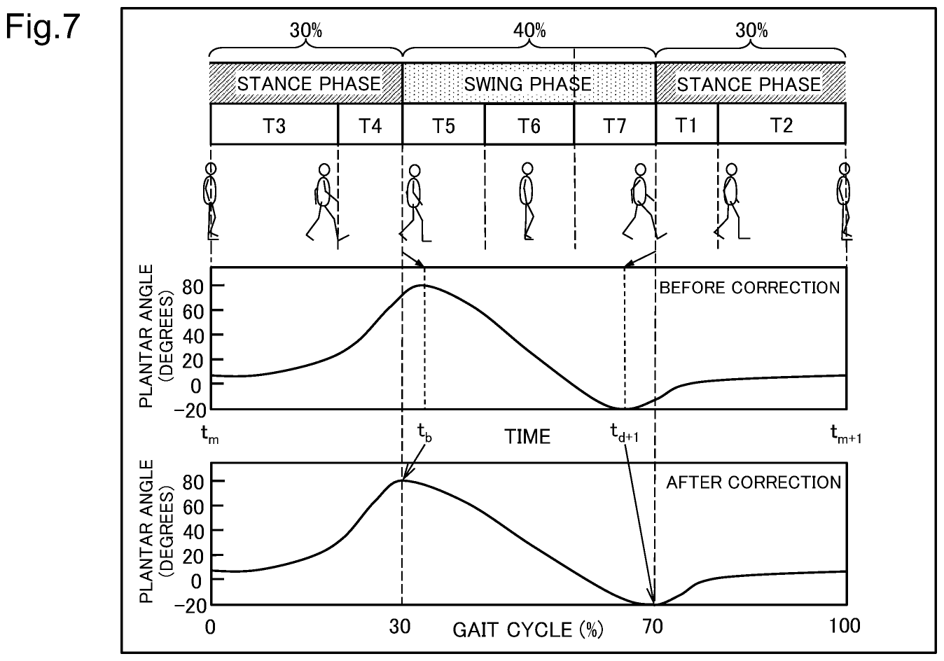

FIG. 7 is a conceptual diagram for explaining the relationship between a gait cycle and time series data of a plantar angle in one gait cycle actually measured. The upper row expresses one gait cycle with time tₘ in the middle of the stance phase as a start point and with time tₘ₊₁ in the middle of the next stance phase as an end point. The graph in the middle row is time series data for one gait cycle of the plantar angle. The horizontal axis of the graph in the middle row is the time when the sensor data for calculating the plantar angle is actually measured, and deviates from the gait cycle of the upper row. In the present example embodiment, the horizontal axis of the time series data of the plantar angle is corrected in order to match the gait cycle.

The anomaly detection device 12 detects, from the time series data of the plantar angle, dorsiflexion peak time t_{d} at which the plantar angle is minimum (dorsiflexion peak) and plantarflexion peak time t_{b} at which the plantar angle is maximum (plantarflexion peak) next to the dorsiflexion peak. Moreover, the anomaly detection device 12 detects dorsiflexion peak time t_{d+1} of the next dorsiflexion peak of the plantarflexion peak and plantarflexion peak time t_{b+1} of the next dorsiflexion peak. The anomaly detection device 12 cuts out gait waveform data for one gait cycle with the time tₘ, which is in the middle between the dorsiflexion peak time t_{d} and the plantarflexion peak time t_{b}, as the start point and with the time tₘ₊₁, which is in the middle between the dorsiflexion peak time t_{d+1} and the plantarflexion peak time t_{b+1}, as the end point. As in FIG. 7, in the gait waveform data for one gait cycle cut out by the anomaly detection device 12, the maximum (plantarflexion peak) appears at the plantarflexion peak time t_{b}, and the minimum (dorsiflexion peak) appears at the dorsiflexion peak time t_{d+1}.

The anomaly detection device 12 normalizes the section from the time tₘ to the time t_{b} to be 30% of the gait cycle, the section from the time t_{b} to the time t_{d+1} to be 40% of the gait cycle, and the section from the time t_{d+1} to the time tₘ₊₁ to be 30% of the gait cycle. The graph in the lower row is the corrected gait waveform data of the plantar angle. The gait waveform data of the plantar angle indicates a change in the plantar angle associated with the gait cycle.

Hereinafter, also regarding time series data of space acceleration and space angular velocity, similarly to the plantar angle, gait waveform data in which the horizontal axis is corrected to the gait cycle will be indicated. 30% of the gait cycle is associated to the timing of the toe off in (e) of FIG. 6. 70% of the gait cycle is associated to the timing of the heel contact in (a) and (g) of FIG. 6.

The anomaly detection device 12 estimates an anomaly in the foot of a pedestrian by using a learned model in which machine learning has been performed using training data where the progression state of the anomaly in the foot is used as a label and a feature amount of a feature site of gait waveform data obtained according to the walking of the pedestrian having the anomaly in the foot is used as input data. Specifically, the anomaly detection device 12 estimates the progression state of hallux valgus of a pedestrian by using a learned model in which machine learning has been performed using training data where the progression state of hallux valgus is used as a label and the feature amount of the feature site of the gait waveform data obtained in response to the walking of the pedestrian in the progression state is used as input data. For example, the anomaly detection device 12 inputs the feature amount of the feature site of the gait waveform data to the learned model, and estimates the HV angle of the foot of the pedestrian. The anomaly detection device 12 outputs the estimated progression state of hallux valgus. A learned model used by the anomaly detection device 12 to estimate the progression state of hallux valgus will be described later.

[Data Acquisition Device]

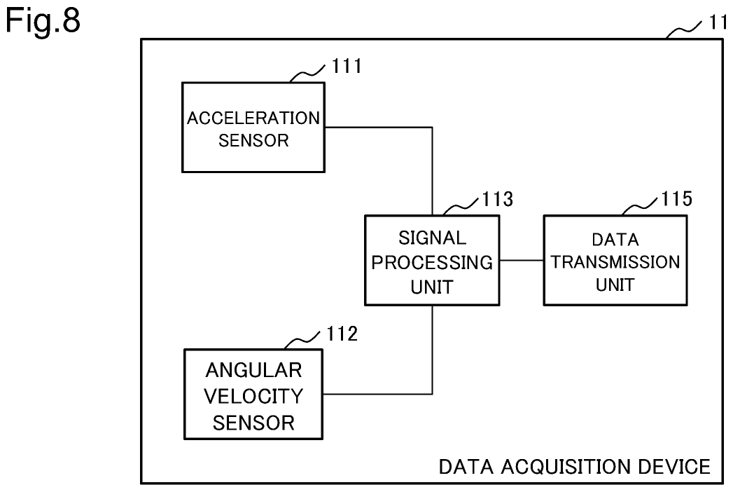

Next, details of the data acquisition device 11 included in the determination system 1 will be described with reference to the drawings. FIG. 8 is a block diagram illustrating an example of the configuration of the data acquisition device 11. The data acquisition device 11 has an acceleration sensor 111, an angular velocity sensor 112, a signal processing unit 113, and a data transmission unit 115.

The acceleration sensor 111 is a sensor that measures the acceleration in the three axial directions. The acceleration sensor 111 outputs the measured acceleration to the signal processing unit 113.

The angular velocity sensor 112 is a sensor that measures the angular velocity in the three axial directions. The angular velocity sensor 112 outputs the measured angular velocity to the signal processing unit 113.

The signal processing unit 113 acquires acceleration and angular velocity from the acceleration sensor 111 and the angular velocity sensor 112, respectively. The signal processing unit 113 converts the acquired acceleration and angular velocity into digital data, and outputs the converted digital data (also referred to as sensor data) to the data transmission unit 115. The sensor data at least includes acceleration data (including acceleration vectors in the three axial directions) in which acceleration of analog data is converted into digital data and angular velocity data (including angular velocity vectors in the three axial directions) in which angular velocity of analog data is converted into digital data. The acceleration data and the angular velocity data are associated with acquisition time of them. The signal processing unit 113 may be configured to output, to the acquired acceleration data and angular velocity data, sensor data to which corrections such as a mounting error, temperature correction, and linearity correction are added.

The data transmission unit 115 acquires sensor data from the signal processing unit 113. The data transmission unit 115 transmits the acquired sensor data to the anomaly detection device 12. The data transmission unit 115 may transmit the sensor data to the anomaly detection device 12 via a wire such as a cable, or may transmit the sensor data to the anomaly detection device 12 via wireless communication. For example, the data transmission unit 115 can be configured to transmit sensor data to the anomaly detection device 12 via a wireless communication function (not illustrated) conforming to a standard such as Bluetooth (registered trademark) or WiFi (registered trademark). The communication function of the data transmission unit 115 may conform to a standard other than Bluetooth (registered trademark) or WiFi (registered trademark).

[Anomaly Detection Device]

Next, details of the anomaly detection device 12 included in the determination system 1 will be described with reference to the drawings. FIG. 9 is a block diagram illustrating an example of the configuration of the anomaly detection device 12. The anomaly detection device 12 includes the extraction unit 121 and the detection unit 123.

The extraction unit 121 acquires sensor data from the data acquisition device 11 (sensor) installed in the footwear. The extraction unit 121 uses the sensor data to extract a gait feature amount characteristic in gait of the pedestrian wearing the footwear.

For example, the extraction unit 121 acquires three-dimensional acceleration data and angular velocity data in the local coordinate system of the data acquisition device 11. The extraction unit 121 converts the acquired sensor data into those in the world coordinate system to generate time series data. For example, the extraction unit 121 generates time series data of three-dimensional acceleration data or time series data of three-dimensional angular velocity data converted into the world coordinate system.

For example, the extraction unit 121 generates time series data such as space acceleration and space angular velocity. The extraction unit 121 integrates the space acceleration and the space angular velocity, and generates time series data of the space velocity and the space angle (plantar angle). The extraction unit 121 generates time series data at a predetermined timing or time interval having been set in accordance with a general gait cycle or a gait cycle unique to the user. The timing at which the extraction unit 121 generates time series data can be discretionarily set. For example, the extraction unit 121 continues to generate time series data during a period in which gait of the user is continued. The extraction unit 121 may be configured to generate time series data at a specific time.

For example, the extraction unit 121 extracts time series data for one gait cycle from generated time series data. The extraction unit 121 generates waveform data (hereinafter, referred to as gait waveform data) for one gait cycle in which time series data for one gait cycle is caused to be associated to the gait cycle. The gait waveform data generated by the extraction unit 121 will be described in detail later.

For example, the extraction unit 121 extracts the feature amount (gait feature amount) of the feature site from the gait waveform data. For example, the extraction unit 121 extracts the gait feature amount from the time series data of the angular velocity (roll angular velocity) about the X axis, the acceleration (Z direction acceleration) in the gravity direction, and the acceleration (Y direction acceleration) in the traveling direction.

The detection unit 123 detects an anomaly in the foot of the pedestrian walking wearing the footwear based on the gait feature amount extracted by the extraction unit 121. For example, the detection unit 123 stores a learned model in which machine learning has been performed using training data where the progression state of the anomaly in the foot is used as a label and a gait feature amount of gait waveform data obtained according to the walking of the pedestrian having the anomaly in the foot is used as input data. In that case, the detection unit 123 inputs the gait feature amount extracted by the extraction unit 121 to the learned model, estimates the progression state of the anomaly in the foot of the pedestrian, and outputs a determination result regarding the estimated progression state of the anomaly in the foot. For example, the detection unit 123 outputs the determination result regarding the progression state of the anomaly in the foot to a system that distributes content according to the determination result or an output device such as a display device or a printing device that is not illustrated.

For example, the detection unit 123 uses a learned model that outputs a determination result indicating whether it is hallux valgus and the range and value of the HV angle. The detection unit 123 outputs the progression state of hallux valgus of the pedestrian by inputting the gait feature amount extracted from the gait waveform data of the pedestrian to the learned model. The detection unit 123 outputs the determination result indicating whether it is hallux valgus and the range and value of the HV angle as the progression state of hallux valgus.

For example, the detection unit 123 uses a learned model that outputs information regarding the progression state of hallux valgus in response to the input of the gait feature amount extracted from the gait waveform data regarding the gait parameter. For example, the detection unit 123 stores in advance a learned model with which a learning device has performed machine learning using training data in which a gait feature amount labeled with identification information regarding the progression state of hallux valgus is used as input data. For example, the learned model can be generated using a method of supervised learning such as a neural network, a support vector machine, a decision tree, and regression. Alternatively, the learned model can be generated using unsupervised learning such as clustering. The learned model may be generated by the determination system 1 or may be generated outside the determination system 1.

For example, the detection unit 123 stores a learned model in which machine learning has been performed using training data where the HV angle is used as a label and the feature amount of the feature site of the gait waveform data obtained according to the walking of the pedestrian with the HV angle is used as input data. The detection unit 123 inputs the gait feature amount extracted by the extraction unit 121 to the learned model, and estimates the HV angle of the foot of the pedestrian.

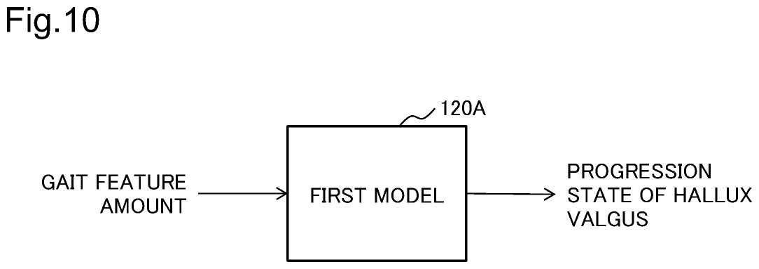

FIG. 10 is a conceptual diagram illustrating an example in which the gait feature amount of gait waveform data is input to a first model 120A in which machine learning has been performed using training data where the progression state of hallux valgus in the foot of a pedestrian is used as a label and the gait feature amount of gait waveform data obtained according to the walking of the pedestrian in the progression state is used as input data. In the example of FIG. 10, in response to the input of the gait feature amount to the first model 120A, the progression state of hallux valgus according to the gait feature amount is output. FIG. 10 illustrates an example in which one gait feature amount is used, but a plurality of gait feature amounts may be used. Use of the first model 120A makes it possible to achieve a service in which, for example, the HV angle is transmitted to a distribution system that distributes content related to gait, and content according to the progression state of hallux valgus is transmitted from the distribution system to the terminal of the pedestrian. The content according to the progression state of hallux valgus may be stored in the terminal of the pedestrian or may be received via a network.

For example, when the HV angle exceeds 20 degrees, the detection unit 123 determines that it is hallux valgus. For example, when the HV angle exceeds a predetermined threshold value of less than 20 degrees, the detection unit 123 determines that there is a tendency of hallux valgus. For example, the detection unit 123 accumulates the estimated HV angle and determines the tendency of hallux valgus according to a change in the accumulated HV angle. For example, when the change in the HV angle tends to increase, the detection unit 123 determines that there is a risk of progressing to hallux valgus. The detection unit 123 outputs a determination result regarding the progression state of hallux valgus.

A person who is insufficient in formation of the arch of foot tends to have a strong impact on the sole during gait. The person insufficient in formation of the arch of foot tends to have an angular velocity about the X axis, an acceleration in the Z direction, an acceleration in the Y direction, and the like that are larger than those of a pedestrian who is less likely to have hallux valgus, for example. For this reason, by walking wearing tight footwear that does not fit the foot, the person with insufficient arch formation is more likely to receive an impact on the thumb and turn to be a hallux valgus. The arch formed in the sole includes a longitudinal arch in a direction along the center line of the foot and a lateral arch in a direction perpendicular to the center line of the foot. In particular, it is inferred that if an impact applied to the lateral arch tends to be strong while walking, a force in a direction where the HV angle increases is easily applied to the thumb, and therefore it tends to be hallux valgus.

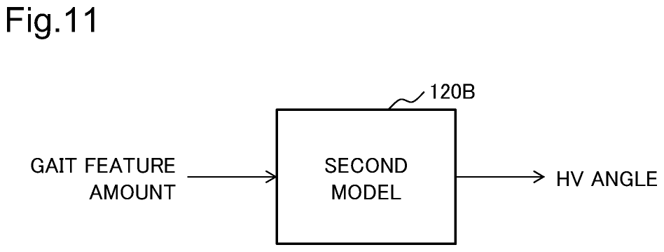

FIG. 11 is a conceptual diagram illustrating an example in which the gait feature amount of gait waveform data is input to a second model 120B in which machine learning has been performed using training data where the HV angle of the foot of the pedestrian is used as a label and the gait feature amount of gait waveform data obtained according to the walking of the pedestrian having the HV angle is used as input data. In the example of FIG. 11, in response to the input of the gait feature amount to the second model 120B, the HV angle according to the gait feature amount is output. FIG. 11 illustrates an example in which one gait feature amount is used, but a plurality of gait feature amounts may be used. Use of the second model 120B makes it possible to achieve a service in which, for example, the HV angle is transmitted to a distribution system that distributes content related to gait, and content according to the HV angle is transmitted from the distribution system to the terminal of the pedestrian. The content according to the HV angle may be stored in the terminal of the pedestrian or may be received via a network.

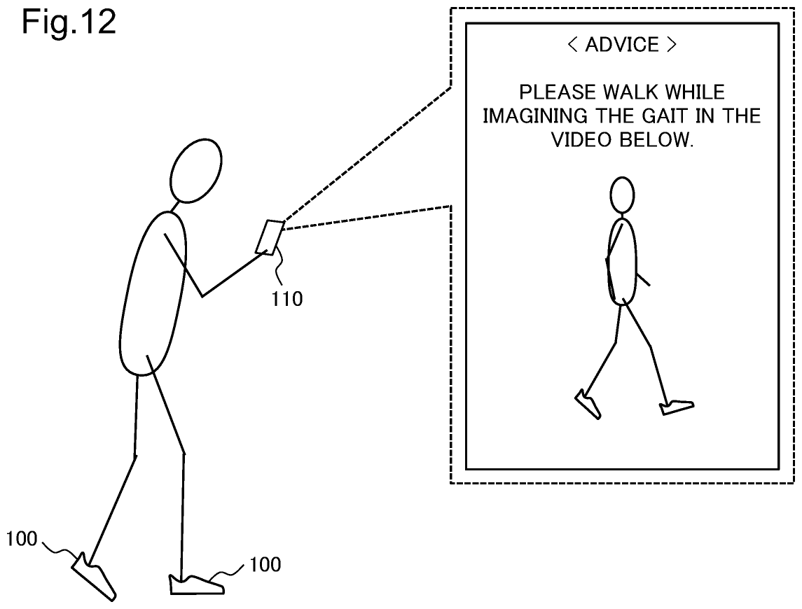

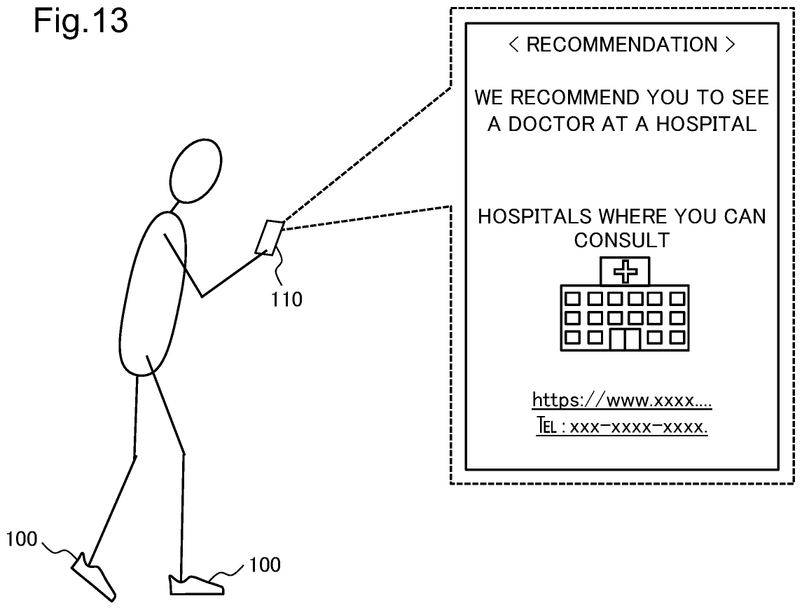

FIGS. 12 and 13 are examples in which content according to the progression state of hallux valgus and the HV angle is displayed on a screen of a mobile terminal 110 of a pedestrian wearing the shoe 100 in which the data acquisition device 11 is installed. However, the mobile terminal 110 is assumed to include the anomaly detection device 12.

FIG. 12 is an example in which a moving image including an ideal gait according to the estimated progression state of hallux valgus and the HV angle is displayed on the mobile terminal 110 of the pedestrian. For example, if the gait of the pedestrian can be measured using the gait waveform data of the pedestrian, advice regarding the gait and posture according to the progression state of hallux valgus and the HV angle may be displayed on the mobile terminal 110 of the pedestrian.

FIG. 13 is an example in which information according to the estimated progression state of hallux valgus and HV angle is displayed on the mobile terminal 110 of the pedestrian. For example, information on recommendation to the pedestrian to see a doctor in a hospital is displayed on the screen of the mobile terminal 110 according to the progression state of hallux valgus and the HV angle. For example, information on hospitals where the pedestrian can consult is displayed on the screen of the mobile terminal 110 according to the progression state of hallux valgus and the HV angle. For example, a link to a website or a telephone number of a hospital where the pedestrian can consult may be displayed on the screen of the mobile terminal 110 according to the progression state of hallux valgus and the HV angle.

[Gait Feature Amount]

Next, as to which feature site of the gait waveform data to extract from according to the extraction of the gait feature amount from the gait waveform data will be explained. Hereinafter, the results in which 51 subjects were recruited for verification of differences in the gait feature amount according to the presence or absence of hallux valgus and the HV angle will be described. In this verification, the subjects were divided into a set (first set) of subjects having an HV angle of more than 20 degrees and a set (second set) of subjects having an HV angle of less than 20 degrees.

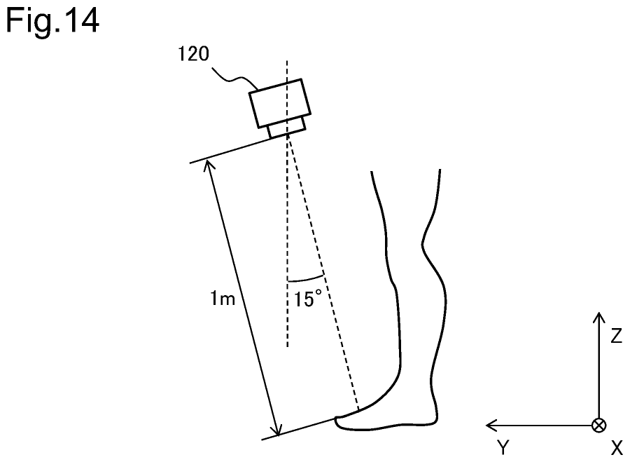

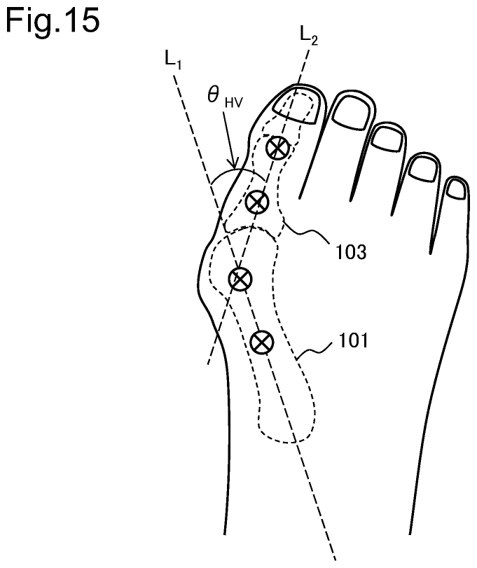

FIGS. 14 and 15 are conceptual diagrams for explaining conditions for measuring the HV angle of the subject.

FIG. 14 is a conceptual diagram for explaining a photographing condition of a camera 120 used for the measurement of the HV angle of the subject. The camera 120 was installed at a position 1 meter (m) from the instep in such a way that the orientation inclined by 15 degrees from the direction (Z direction) perpendicular to the ground (XY plane) was the photographing direction.

FIG. 15 is a conceptual diagram illustrating an example in which the positions of the first metatarsal bone 101 and the first proximal phalanx 103 (dotted line) are extracted from an image photographed by the camera 120, and the HV angle θ_{HV}, which is the angle formed by the center line L₁ of the first metatarsal bone 101 and the center line L₂ of the first proximal phalanx 103, is measured. In the present example embodiment, two protrusion sites of the instep caused by each of the first metatarsal bone 101 and the first proximal phalanx 103 were extracted. Then, an acute angle formed by intersecting a straight line passing through two points extracted from the first metatarsal bone 101 and a straight line passing through two points extracted from the first proximal phalanx 103 was defined as the HV angle θ_{HV}.

On the inside of the footwear worn by the subject, the data acquisition device 11 was positioned below the arch of foot. Then, the gait waveform data for one gait cycle was extracted by using the sensor data obtained according to the walking of the subject wearing the footwear in which the data acquisition device 11 was disposed. The gait waveform data obtained based on gait of the subject was averaged for each subject. For all subjects, the gait waveform data of each of the first set and the second set was averaged. Hereinafter, an example of comparing a mean of all the gait waveform data of the first set with a mean of all the gait waveform data of the second set will be described. Hereinafter, the mean of all the gait waveform data of the first set is referred to as gait waveform data of the first set, and a mean of all the gait waveform data of the second set is referred to as gait waveform data of the second set.

Whether or not there was a difference in the feature amounts of the feature sites extracted from the gait waveform data of the first set and the second set was tested. In the present test, a null hypothesis that there is not a difference in the feature amounts of the feature sites extracted from the gait waveform data of the first set and the second set was made. Among the feature amounts of the feature sites extracted from the gait waveform data of the first set, the feature amount of the feature site having a significant difference from the feature amount of the feature site extracted from the gait waveform data of the second set was defined as a gait feature amount.

<Roll Angular Velocity>

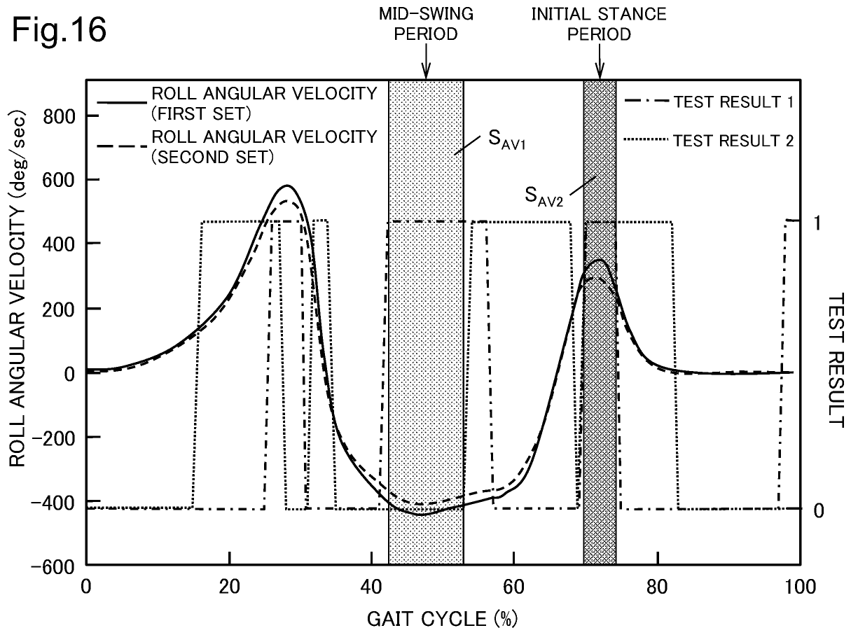

FIG. 16 is gait waveform data of the angular velocity (roll angular velocity) about the X axis obtained by gait of the subject wearing the footwear in which the data acquisition device 11 is disposed (left vertical axis). The gait waveform data of the set (first set) of subjects having an HV angle of more than 20 degrees is indicated by a solid line. The gait waveform data of the set (second set) of subjects having an HV angle of less than 20 degrees is indicated by a broken line.

FIG. 16 illustrates a test result 1 in which it was tested by a t-test (one-dot chain line) whether there was a difference in the feature amounts of the feature sites extracted from the gait waveform data of the first set and the second set in addition to the gait waveform data. For the test result 1, the significance probability that there is not a difference in the feature amounts of the feature sites extracted from the gait waveform data of the first set and the second set is 1 in a case of less than a significance level 0.05, and is 0 otherwise. That is, when the test result 1 is 1, it is significant that there is a difference in the feature amounts of the feature sites extracted from the gait waveform data of the first set and the second set.

Furthermore, FIG. 16 illustrates a test result 2 in which it was tested by a t-test (dotted line) whether there was a correlation between the gait speed (Y direction speed) and the roll angular velocity. The correlation between the gait speed and the roll angular velocity is verified in order to verify whether the feature amount of the feature site extracted from the gait waveform data of the first set is affected by the gait speed. For the test result 2, the significance probability that there is not a correlation between the gait speed and the roll angular velocity is 1 in a case of less than a significance level 0.05, and is 0 otherwise. That is, when the test result 2 is 1, it is significant that there is a correlation between the gait speed and the roll angular velocity. Specifically, a difference in Pearson's product-moment correlation coefficient (hereinafter, also referred to as correlation coefficient) between the gait speed and the roll angular velocity was verified. The gait speed was calculated by dividing a value obtained by integrating the acceleration (Y direction acceleration) in the traveling direction in one gait cycle by time of one gait cycle.

By comparing the gait waveform data of the roll angular velocities of the first set and the second set, a difference in the feature amounts of the feature sites extracted from the gait waveform data of the first set and the second set was significant, and two feature sites associated with the gait feature were extracted (section S_{AV1}, section S_{AV2}).

The section S_{AV1} from the gait cycle about over 40% to about over 50% includes the timing of a mid-swing period. In the section S_{AV1}, the test result 1 is 1, and the test result 2 is 0. That is, the feature amount of the feature site in the section S_{AV1} is not affected by the gait speed. Therefore, the gait feature amount of the feature site extracted from the section S_{AV1} can be used as it is. For example, the gait feature amount extracted from the gait waveform data of the roll angular velocity when the gait cycle is 50% can be used.

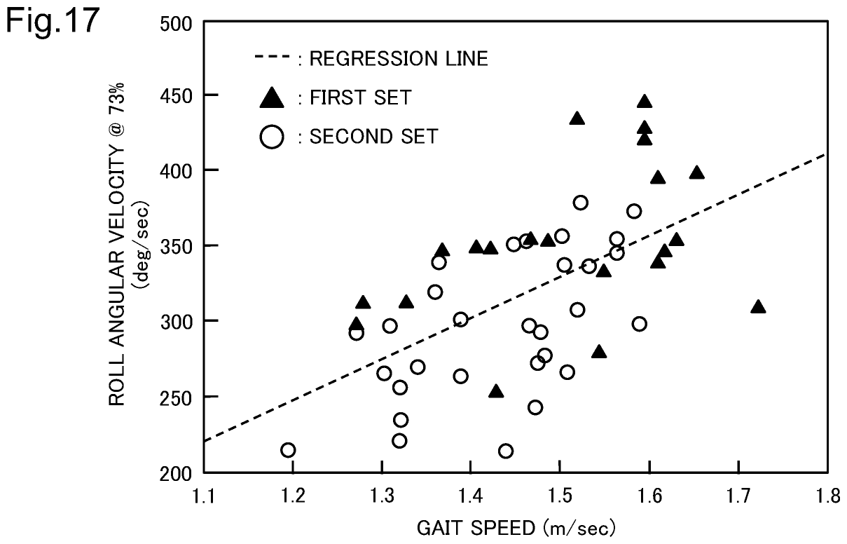

The section S_{AV2} in which the gait cycle is about over 70% includes the timing at an initial stance period. In the section S_{AV2}, the test result 1 is 1, and the test result 2 is also 1. That is, the feature amount of the feature site in the section S_{AV2} is likely to have been affected by the gait speed. Therefore, as illustrated in FIGS. 17 to 19, after the influence of the gait speed was removed from the gait feature amount of the feature site extracted from the section S_{AV2}, it was tested by the t-test whether there was a difference in the feature amounts of the feature sites extracted from the gait waveform data of the first set and the second set.

FIG. 17 is a graph obtained by plotting, with respect to the gait speed, the roll angular velocity when the gait cycle included in the section S_{AV2} is 73%. The graph of FIG. 17 illustrates a regression line (broken line) when the relationship between the gait speed when the gait cycle is 73% and the roll angular velocity at that time is linearly regressed for all the subjects.

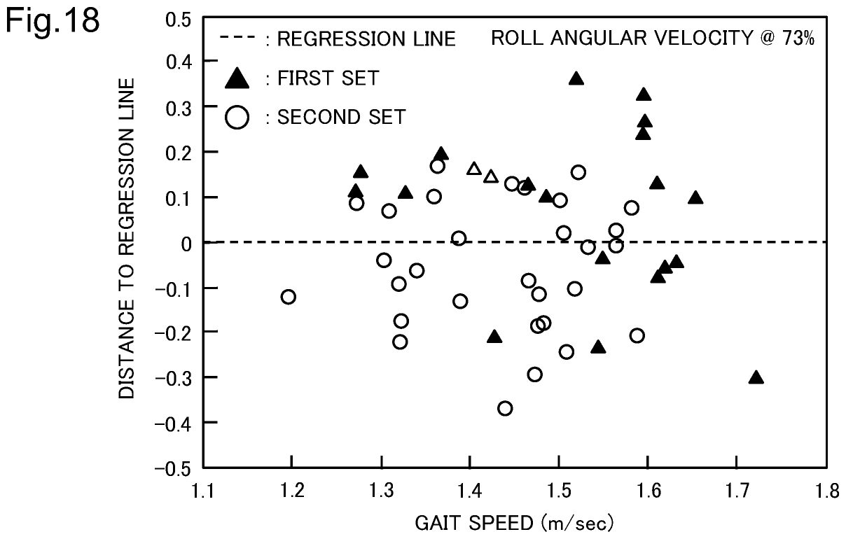

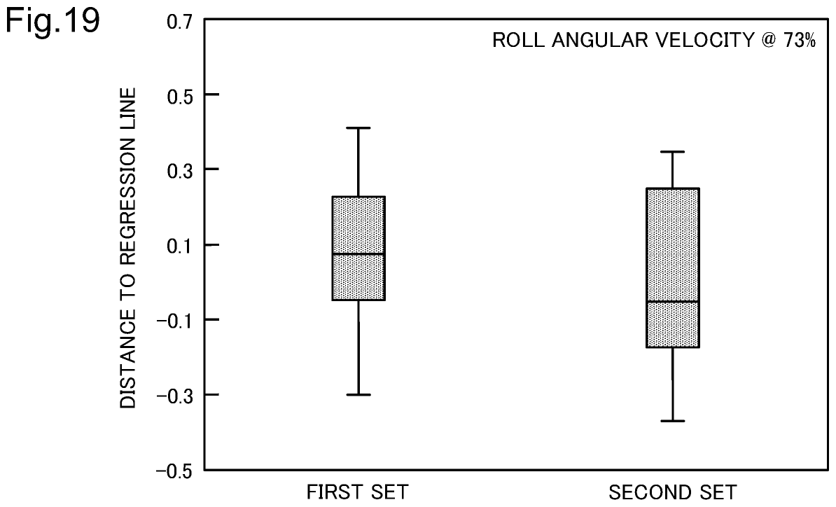

FIG. 18 is a graph obtained by plotting, with respect to the gait speed when the gait cycle is 73%, the distance between the roll angular velocity and the regression line when the gait cycle is 73%. In FIG. 19, the sign of the distance of the plot above the regression line is set to plus, and the sign of the distance of the plot below the regression line is set to minus.

FIG. 19 is a box-and-whisker diagram regarding the distance between the roll angular velocity and the regression line when the gait cycle is 73%. Regarding the roll angular velocity when the gait cycle was 73%, the set (first set) of the subjects having the HV angle of more than 20 degrees had a smaller interquartile range (variation) and a larger median. Regarding the roll angular velocity when the gait cycle was 73%, when the influence of the gait speed was removed, the significance probability that there is not a difference in the feature amounts of the feature sites extracted from the gait waveform data of the first set and the second set was less than the significance level 0.05. That is, regarding the roll angular velocity when the gait cycle is 73%, it is significant that there is a difference in the feature amounts of the feature sites extracted from the gait waveform data of the first set and the second set. Therefore, the feature amount extracted from the roll angular velocity at the gait cycle of 73% can be used as the gait feature amount for extracting the set (first set) of the subjects having the HV angle of more than 20 degrees.

That is, regarding the roll angular velocity, as the gait feature amount for extracting the set (first set) of the subjects having the HV angle of more than 20 degrees, the feature amount of the feature site extracted from each of the section S_{AV1} included in the mid-swing period and the section S_{AV2} included in the initial stance period can be used. It is desirable to remove the influence of the gait speed from the feature amount of the feature site extracted from the section S_{AV2} included in the initial stance period. In a case where the roll angular velocity is used as a gait parameter, for example, a feature amount extracted from a feature site in the vicinity of a gait cycle of 50% or 73% can be used as a gait feature amount for extracting the set (first set) of the subjects having the HV angle of more than 20 degrees.

<Z Direction Acceleration>

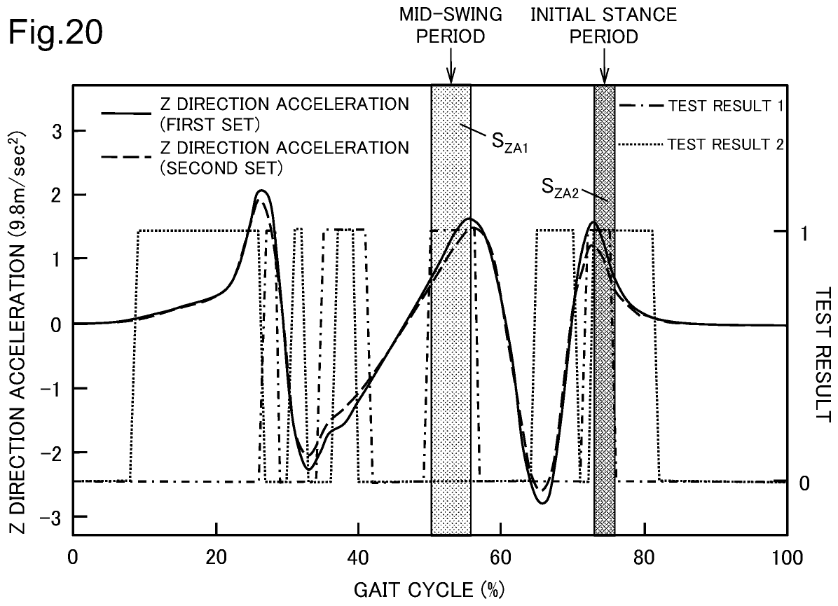

FIG. 20 is gait waveform data of the Z direction acceleration obtained by gait of the subject wearing the footwear in which the data acquisition device 11 is disposed (left vertical axis). The gait waveform data of the set (first set) of subjects having an HV angle of more than 20 degrees is indicated by a solid line. The gait waveform data of the set (second set) of subjects having an HV angle of less than 20 degrees is indicated by a broken line.

FIG. 20 illustrates the test result 1 in which it was tested by a t-test (one-dot chain line) whether there was a difference in the feature amounts of the feature sites extracted from the gait waveform data of the first set and the second set in addition to the gait waveform data. For the test result 1, the significance probability that there is not a difference in the feature amounts of the feature sites extracted from the gait waveform data of the first set and the second set is 1 in a case of less than a significance level 0.05, and is 0 otherwise. That is, when the test result 1 is 1, it is significant that there is a difference in the feature amounts of the feature sites extracted from the gait waveform data of the first set and the second set.

Furthermore, FIG. 20 illustrates the test result 2 in which it was tested by a t-test (dotted line) whether there was a correlation between the gait speed (Y direction speed) and the Z direction acceleration. The correlation between the gait speed and the Z direction acceleration is verified in order to verify whether the feature amount of the feature site extracted from the gait waveform data of the first set is affected by the gait speed. For the test result 2, the significance probability that there is not a correlation between the gait speed and the Z direction acceleration is 1 in a case of less than a significance level 0.05, and is 0 otherwise. That is, when the test result 2 is 1, it is significant that there is a correlation between the gait speed and the Z direction acceleration. Specifically, a difference in Pearson's product-moment correlation coefficient (hereinafter, also referred to as correlation coefficient) between the gait speed and the Z direction acceleration was verified. The gait speed was calculated by dividing a value obtained by integrating the acceleration (Y direction acceleration) in the traveling direction in one gait cycle by time of one gait cycle.

By comparing the gait waveform data of the Z direction accelerations of the first set and the second set, a difference in the feature amounts of the feature sites extracted from the gait waveform data of the first set and the second set was significant, and two feature sites associated with the gait feature were extracted (section S_{ZA1}, section S_{ZA2}).

The section S_{ZA1} of the gait cycle about over 50% includes the timing of a mid-swing period. In the section S_{ZA1}, the test result 1 is 1, and the test result 2 is 0. Therefore, the gait feature amount of the feature site extracted from the section S_{ZA1} can be used as it is. For example, the gait feature amount extracted from the gait waveform data of the Z direction acceleration when the section S_{ZA1} is 52% can be used.

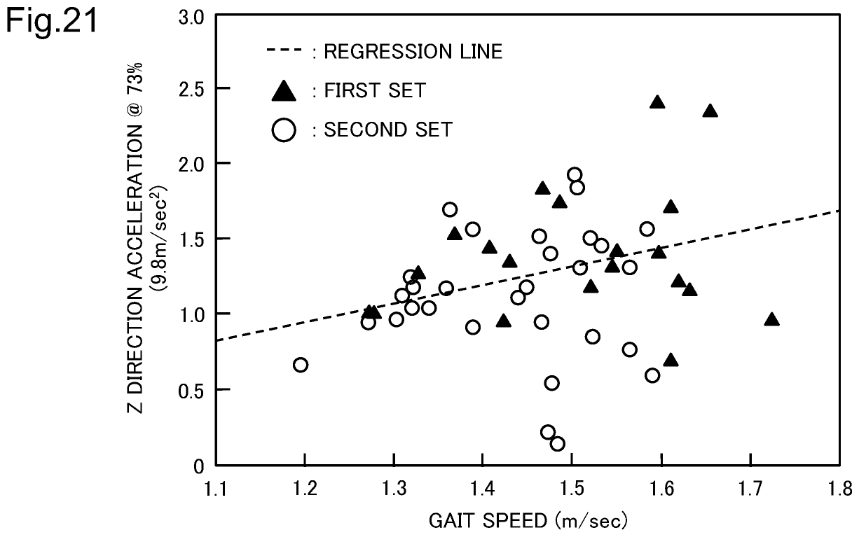

The section S_{ZA2} in which the gait cycle is between 70% and 80% includes the timing of heel rocker included in the initial stance period. In the section S_{ZA2}, the test result 1 is 1, and the test result 2 is also 1. That is, the feature amount of the feature site in the section S_{ZA2} is likely to have been affected by the gait speed. Therefore, as illustrated in FIGS. 21 to 23, after the influence of the gait speed was removed from the gait feature amount of the feature site extracted from the section S_{ZA2}, it was tested by the t-test whether there was a difference in the feature amounts of the feature sites extracted from the gait waveform data of the first set and the second set.

FIG. 21 is a graph obtained by plotting, with respect to the gait speed, the Z direction acceleration when the gait cycle included in the section S_{ZA2} is 73%. The graph of FIG. 21 illustrates a regression line (broken line) when the relationship between the gait speed when the gait cycle is 73% and the Z direction acceleration at that time is linearly regressed for all the subjects.

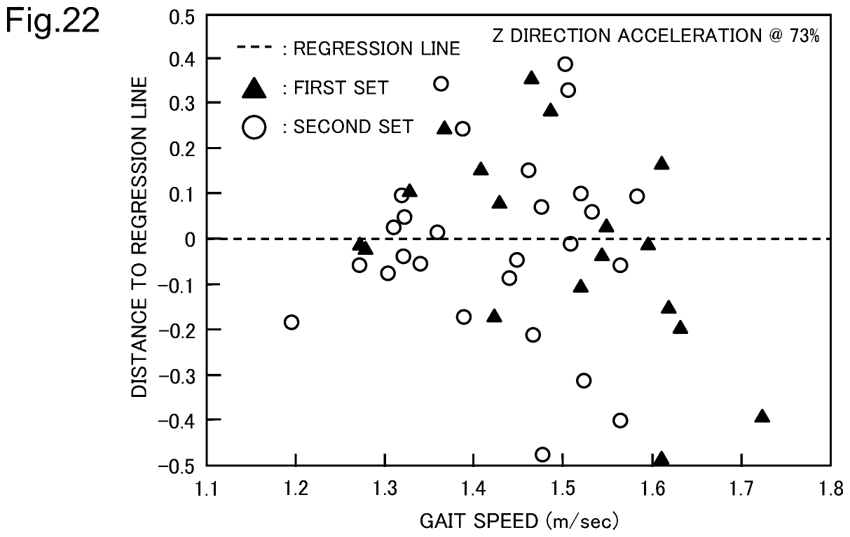

FIG. 22 is a graph obtained by plotting, with respect to the gait speed when the gait cycle is 73%, the distance between the Z direction acceleration and the regression line when the gait cycle is 73%. In FIG. 22, the sign of the distance of the plot above the regression line is set to plus, and the sign of the distance of the plot below the regression line is set to minus.

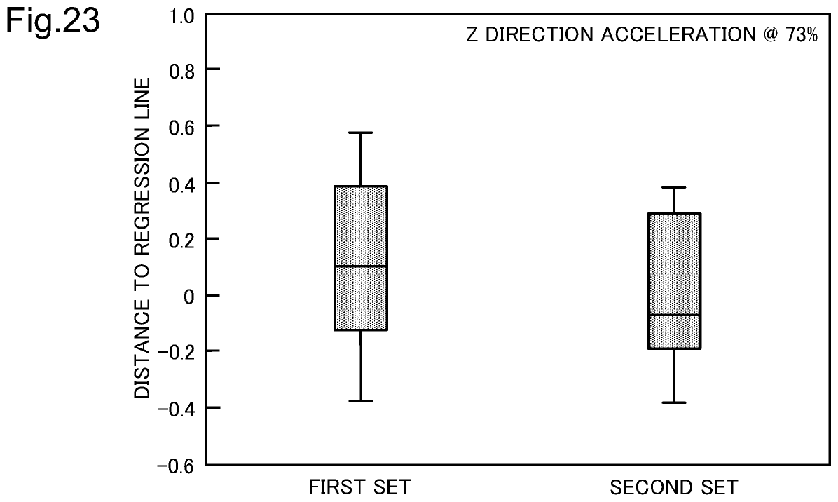

FIG. 23 is a box-and-whisker diagram regarding the distance between the Z direction acceleration and the regression line when the gait cycle is 73%. Regarding the Z direction acceleration when the gait cycle was 73%, the set (first set) of the subjects having the HV angle of more than 20 degrees had a smaller interquartile range (variation) and a larger median. Regarding the Z direction acceleration at the gait cycle of 73%, when the influence of the gait speed was removed, the significance probability that there is not a difference in the feature amounts of the feature sites extracted from the gait waveform data of the first set and the second set was less than the significance level 0.05. That is, regarding the Z direction acceleration when the gait cycle is 73%, it is significant that there is a difference in the feature amounts of the feature sites extracted from the gait waveform data of the first set and the second set. Therefore, the feature amount extracted from the Z direction acceleration when the gait cycle is 73% can be used as the gait feature amount for extracting the set (first set) of the subjects having the HV angle of more than 20 degrees.

That is, regarding the Z direction acceleration, as the gait feature amount for extracting the set (first set) of the subjects having the HV angle of more than 20 degrees, the feature amount of the feature site extracted from each of the section S_{ZA1} included in the mid-swing period and the section S_{ZA2} included in the initial stance period can be used. It is desirable to remove the influence of the gait speed from the feature amount of the feature site extracted from the section S_{ZA2} included in the initial stance period. In a case where the Z direction acceleration is used as a gait parameter, for example, a feature amount extracted from a feature site in the vicinity of a gait cycle of 50% or 73% can be used as a gait feature amount for extracting the set (first set) of the subjects having the HV angle of more than 20 degrees.

<Y Direction Acceleration>

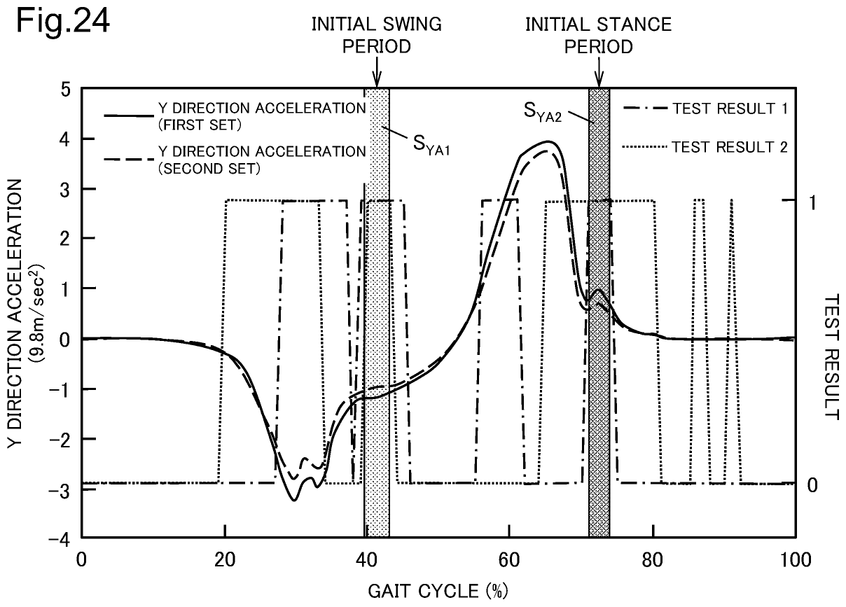

FIG. 24 is gait waveform data of the Y direction acceleration obtained by gait of the subject wearing the footwear in which the data acquisition device 11 is disposed (left vertical axis). The gait waveform data of the set (first set) of subjects having an HV angle of more than 20 degrees is indicated by a solid line. The gait waveform data of the set (second set) of subjects having an HV angle of less than 20 degrees is indicated by a broken line.

FIG. 24 illustrates the test result 1 in which it was tested by a t-test (one-dot chain line) whether there was a difference in the feature amounts of the feature sites extracted from the gait waveform data of the first set and the second set in addition to the gait waveform data. For the test result 1, the significance probability that there is not a difference in the feature amounts of the feature sites extracted from the gait waveform data of the first set and the second set is 1 in a case of less than a significance level 0.05, and is 0 otherwise. That is, when the test result 1 is 1, it is significant that there is a difference in the feature amounts of the feature sites extracted from the gait waveform data of the first set and the second set.

Furthermore, FIG. 24 illustrates the test result 2 in which it was tested by a t-test (dotted line) whether there was a correlation between the gait speed (Y direction speed) and the Y direction acceleration. The correlation between the gait speed and the Y direction acceleration is verified in order to verify whether the feature amount of the feature site extracted from the gait waveform data of the first set is affected by the gait speed. For the test result 2, the significance probability that there is not a correlation between the gait speed and the Y direction acceleration is 1 in a case of less than a significance level 0.05, and is 0 otherwise. That is, when the test result 2 is 1, it is significant that there is a correlation between the gait speed and the Y direction acceleration. Specifically, a difference in Pearson's product-moment correlation coefficient (hereinafter, also referred to as correlation coefficient) between the gait speed and the Y direction acceleration was verified. The gait speed was calculated by dividing a value obtained by integrating the acceleration (Y direction acceleration) in the traveling direction in one gait cycle by time of one gait cycle.

By comparing the gait waveform data of the Y direction accelerations of the first set and the second set, a difference in the feature amounts of the feature sites extracted from the gait waveform data of the first set and the second set was significant, and two feature sites associated with the gait feature were extracted (section S_{YA1}, section S_{YA2}).

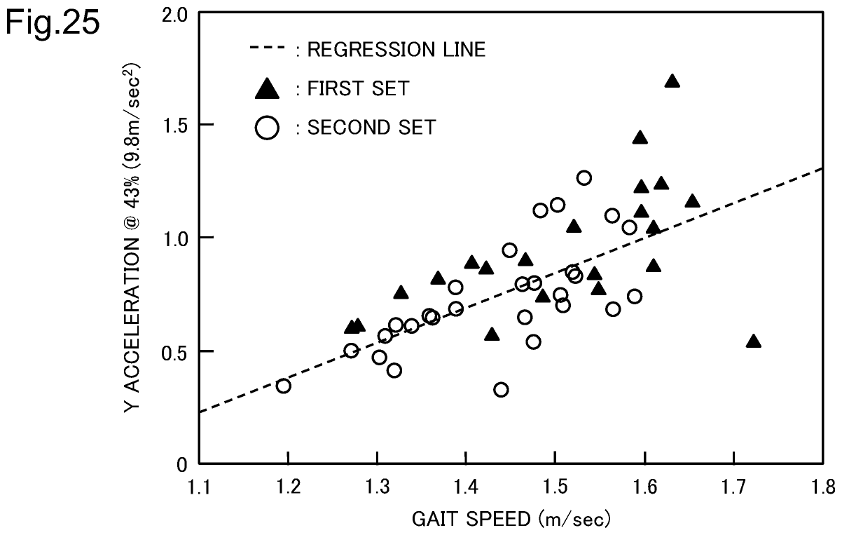

The section S_{YA1} in which the gait cycle is about 40% includes the timing at the initial swing period. The section S_{YA2} in which the gait cycle is about over 70% includes the timing at an initial stance period. In the section S_{YA1} and the section S_{YA2}, the test result 1 is 1, and the test result 2 is also 1. That is, the feature amount of the feature site in the section S_{YA1} and the section S_{YA2} is likely to have been affected by the gait speed. Therefore, as illustrated in FIGS. 25 to 27 and FIGS. 28 to 30, after the influence of the gait speed was removed from the gait feature amounts of the feature sites extracted from the section S_{YA1} and the section S_{YA2}, it was tested by the t-test whether there was a difference in the feature amounts of the feature sites extracted from the gait waveform data of the first set and the second set.

FIG. 25 is a graph obtained by plotting, with respect to the gait speed, the Y direction acceleration when the gait cycle included in the section S_{YA1} is 43%. The graph of FIG. 25 illustrates a regression line (broken line) when the relationship between the Y direction acceleration when the gait cycle is 43% and the gait speed at that time is linearly regressed for all the subjects.

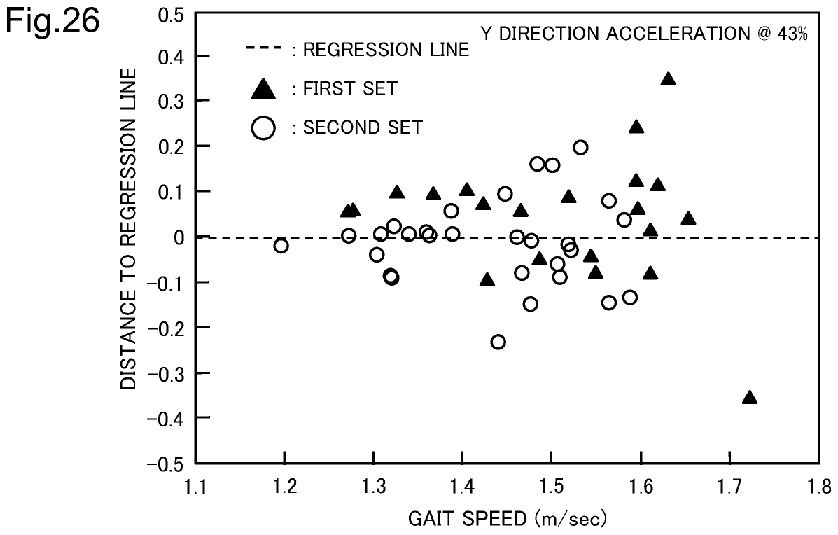

FIG. 26 is a graph obtained by plotting, with respect to the gait speed when the gait cycle is 43%, the distance between the Y direction acceleration and the regression line when the gait cycle is 43%. In FIG. 26, the sign of the distance of the plot above the regression line is set to plus, and the sign of the distance of the plot below the regression line is set to minus.

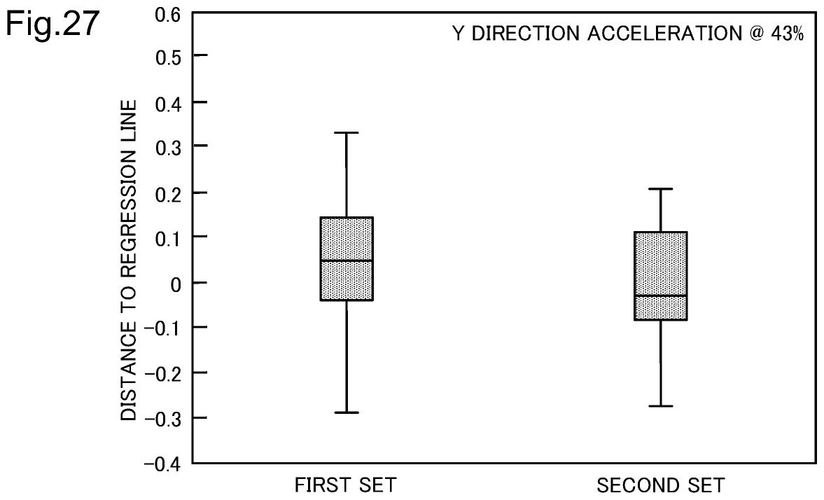

FIG. 27 is a box-and-whisker diagram regarding the distance between the Y direction acceleration and the regression line when the gait cycle is 43%. Regarding the Y direction acceleration when the gait cycle was 43%, the set (first set) of the subjects having the HV angle of more than 20 degrees had a smaller interquartile range (variation) and a larger median. Regarding the Y direction acceleration at the gait cycle of 43%, when the influence of the gait speed was removed, the significance probability that there is not a difference in the feature amounts of the feature sites extracted from the gait waveform data of the first set and the second set was less than the significance level 0.05. That is, regarding the Y direction acceleration when the gait cycle is 43%, it is significant that there is a difference in the feature amounts of the feature sites extracted from the gait waveform data of the first set and the second set. Therefore, the feature amount extracted from the Y direction acceleration when the gait cycle is 43% can be used as the gait feature amount for extracting the set (first set) of the subjects having the HV angle of more than 20 degrees.

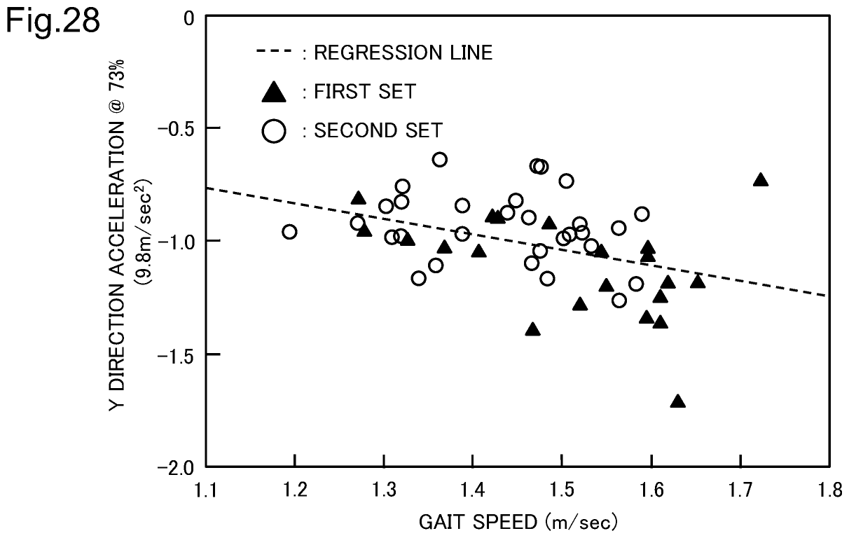

FIG. 28 is a graph obtained by plotting, with respect to the gait speed, the Y direction acceleration when the gait cycle included in the section S_{YA2} is 73%. The graph of FIG. 28 illustrates a regression line (broken line) when the relationship between the Y direction acceleration when the gait cycle is 73% and the gait speed at that time is linearly regressed for all the subjects.

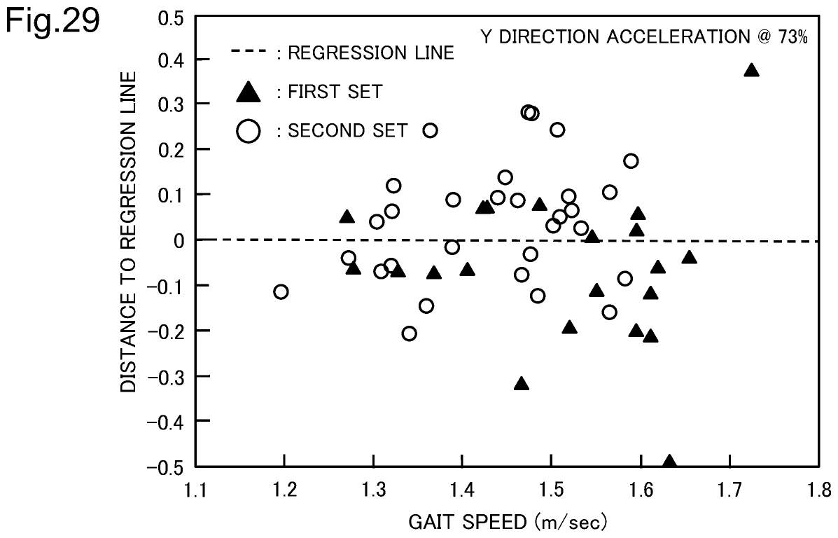

FIG. 29 is a graph obtained by plotting, with respect to the gait speed when the gait cycle is 73%, the distance between the Y direction acceleration and the regression line when the gait cycle is 73%. In FIG. 29, the sign of the distance of the plot above the regression line is set to plus, and the sign of the distance of the plot below the regression line is set to minus.

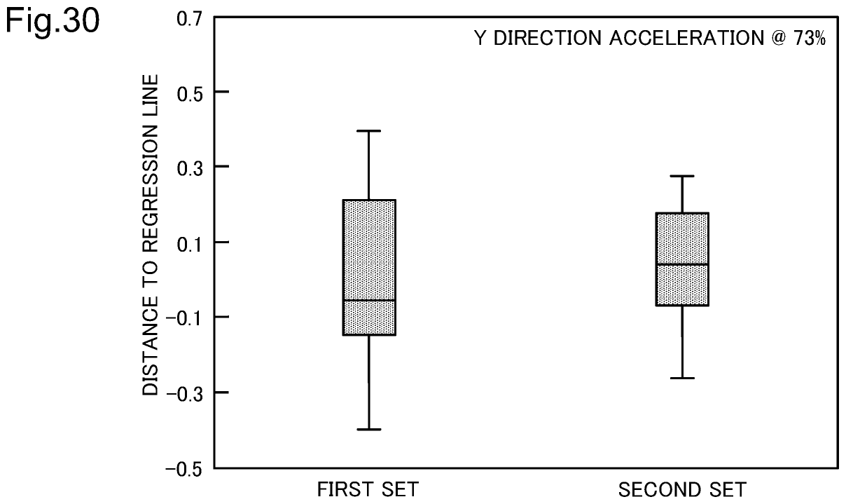

FIG. 30 is a box-and-whisker diagram regarding the distance between the Y direction acceleration and the regression line when the gait cycle is 73%. Regarding the Y direction acceleration when the gait cycle was 73%, the set (first set) of the subjects having the HV angle of more than 20 degrees had a smaller interquartile range (variation) and a larger median. Regarding the Y direction acceleration at the gait cycle of 73%, when the influence of the gait speed was removed, the significance probability that there is not a difference in the feature amounts of the feature sites extracted from the gait waveform data of the first set and the second set was less than the significance level 0.05. That is, regarding the Y direction acceleration when the gait cycle is 73%, it is significant that there is a difference in the feature amounts of the feature sites extracted from the gait waveform data of the first set and the second set. Therefore, the feature amount extracted from the Y direction acceleration when the gait cycle is 73% can be used as the gait feature amount for extracting the set (first set) of the subjects having the HV angle of more than 20 degrees.

That is, regarding the Y direction acceleration, as the gait feature amount for extracting the set (first set) of the subjects having the HV angle of more than 20 degrees, the feature amount of the feature site extracted from each of the section S_{YA1} included in the initial swing period and the section S_{YA2} included in the initial stance period can be used. It is desirable to remove the influence of the gait speed from the feature amount of the feature site extracted from each of the section S_{YA1} included in the initial swing period and the section S_{YA2} included in the initial stance period. In a case where the Y direction acceleration is used as a gait parameter, for example, a feature amount extracted from a feature site in the vicinity of a gait cycle of 43% or 73% can be used as a gait feature amount for extracting the set (first set) of the subjects having the HV angle of more than 20 degrees.

The above is the explanation on as to which feature site of the gait waveform data to extract from when extracting the gait feature amount from the gait waveform data regarding the gait parameters such as the roll angular velocity, the Z direction acceleration, and the Y direction acceleration. Note that the gait parameters used by the anomaly detection device 12 are not limited to the roll angular velocity, the Z direction acceleration, and the Y direction acceleration. As the gait parameters used by the anomaly detection device 12, any gait parameters can be used as long as an anomaly in the foot such as the progression state of hallux valgus can be detected.

(Operation)

Next, the operation of the determination system 1 of the present example embodiment will be described with reference to the drawings. Hereinafter, the extraction unit 121 and the detection unit 123 of the determination system 1 are entities of operations. The entity of the operation described below may be the determination system 1.

[Extraction Unit]

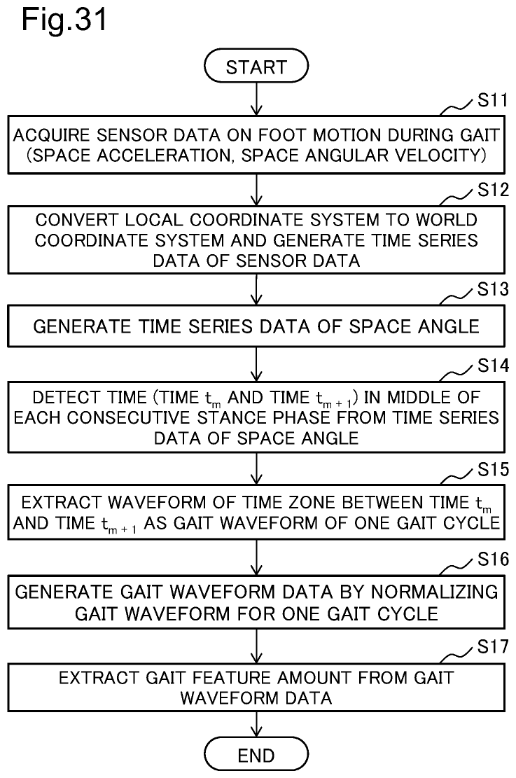

First, the operation of the extraction unit 121 of the determination system 1 will be described with reference to the drawings. FIG. 31 is a flowchart for explaining an example of the operation of the extraction unit 121.

In FIG. 31, first, the extraction unit 121 acquires, from the data acquisition device 11, sensor data regarding the motion of the foot of the pedestrian walking wearing the footwear in which the data acquisition device 11 is installed (step S11). The extraction unit 121 acquires sensor data in the local coordinate system of the data acquisition device 11. For example, as sensor data regarding the motion of the foot, the extraction unit 121 acquires a three-dimensional space acceleration and a three-dimensional space angular velocity from the data acquisition device 11.

Next, the extraction unit 121 converts the coordinate system of the acquired sensor data from the local coordinate system to the world coordinate system, and generates time series data of the sensor data (step S12).

Next, the extraction unit 121 calculates the space angle using at least any of the space acceleration and the space angular velocity, and generates time series data of the space angle (step S13). The extraction unit 121 generates time series data of a space velocity and a spatial trajectory as necessary. Step S13 may be performed before step S12.

Next, the extraction unit 121 detects the time (time tₘ, time tₘ₊₁) in the middle of each of the consecutive stance phases from the time series data of the space angle (step S14).

Next, the extraction unit 121 extracts a waveform of a time zone between the time tₘ and the time tₘ₊₁ as a gait waveform for one gait cycle from the time series data of the space acceleration and the space angular velocity of the extraction target of the gait feature amount (step S15).

Next, the extraction unit 121 normalizes the gait waveform for one gait cycle extracted from the time series data of the space acceleration and the space angular velocity, and generates gait waveform data (step S16). The normalization mentioned here is to correct the gait waveform in such a way that the section from time tₘ to time t_{b} is 30% of the gait cycle, the section from time t_{b} to time t_{d+1} is 40% of the gait cycle, and the section from time t_{d+1} to time tₘ+₁ is 30% of the gait cycle as illustrated in FIG. 7.

Then, the extraction unit 121 extracts the feature amount (gait feature amount) of the feature site from the generated gait waveform data (step S17).

[Detection Unit]

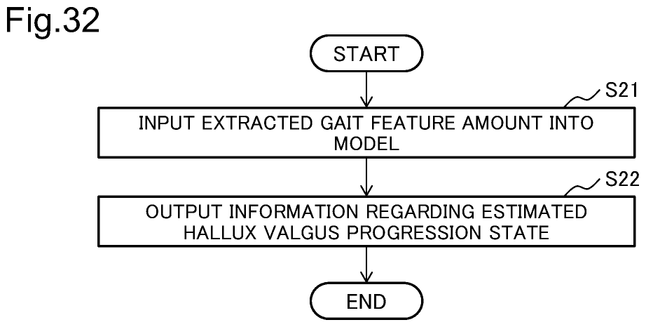

Next, the operation of the detection unit 123 of the determination system 1 will be described with reference to the drawings. FIG. 32 is a flowchart for explaining an example of the operation of the detection unit 123.

In FIG. 32, first, the detection unit 123 inputs the gait feature amount extracted by the extraction unit 121 into a learned model (step S21).

Then, the detection unit 123 outputs information regarding the progression state of hallux valgus based on the output from the learned model (step S22).

The above is the explanation on the operation of the determination system 1. Note that FIGS. 31 and 32 are examples and do not limit the operation of the determination system 1.

<Selection Method of Gait Feature Amount>

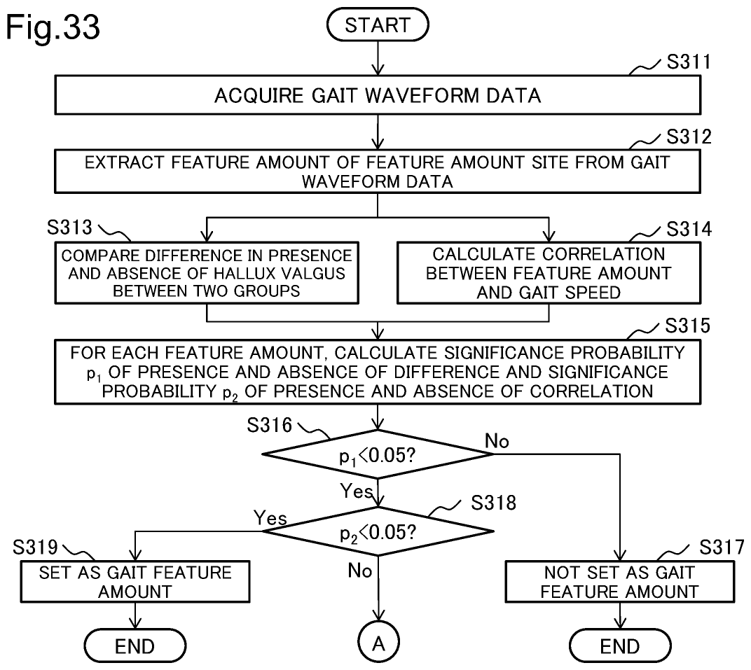

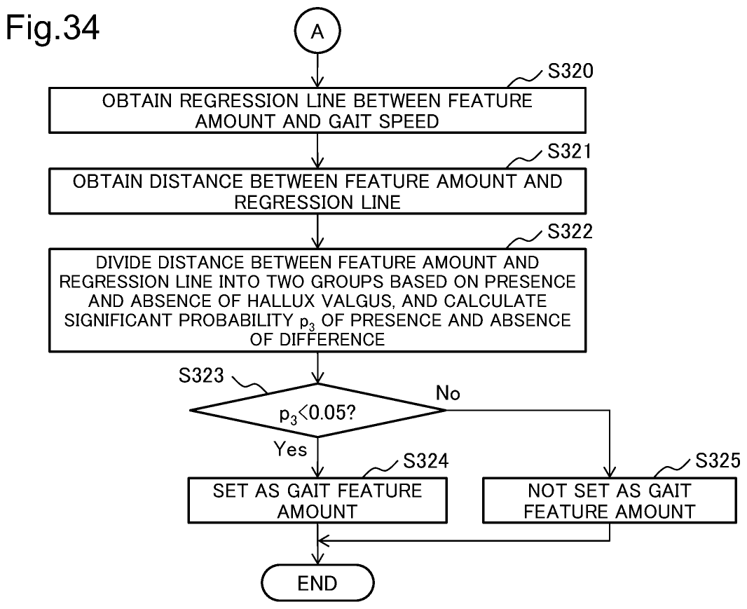

Next, a selection method of the gait feature amount will be described with reference to the drawings. FIGS. 33 and 34 are flowcharts for explaining an example of the selection method of the gait feature amount. Normally, the processing of the determination system 1 does not include selection of the gait feature amount. However, the determination system 1 may be configured to select the gait feature amount. In that case, a selection unit that selects the gait feature amount may be added to the determination system 1. In the following description, it is assumed that the determination system 1 selects the gait feature amount.

In FIG. 33, the determination system 1 acquires normalized gait waveform data (step S311).

Next, the determination system 1 extracts the feature amount of the feature site from the acquired gait waveform data (step S312). After step S312, the determination system 1 performs two processing (step S313, step S314) concurrently. The processing of step S313 and step S314 may be performed sequentially. In a case of sequentially performing the processing of step S313 and step S314, the sequence of executing the processing of step S313 and step S314 is discretionary.

After step S312, as first processing, the determination system 1 calculates the mean of the gait waveform data of the two groups (first set and second set) divided in terms of the presence and absence of hallux valgus, and compares the difference in the mean of the gait waveform data between the two groups (step S313). After step S313, the process proceeds to step S315.

After step S312, as second processing, the determination system 1 calculates the correlation between the feature amount and the gait speed (step S314). After step S314, the process proceeds to step S315.

Next, the determination system 1 calculates, for the feature amount of the feature site extracted from the gait waveform data, a significance probability p₁ of presence and absence of a difference between the two groups and a significance probability p₂ of presence or absence of a correlation between the feature amount and the gait speed (step S315).

If the significance probability p₁ of presence and absence of the difference between the two groups is equal to or more than the significance level 0.05 (No in step S316), there is not a significant difference in the difference between the two groups, and therefore the determination system 1 does not set the feature amount as a gait feature amount (step S317). On the other hand, if the significance probability p₁ of presence and absence of the difference between the two groups is less than the significance level 0.05 (Yes in step S316), there is a significant difference in the difference between the two groups, and therefore the process proceeds to step S318.

If the significance probability p₂ of presence or absence of a correlation between the feature amount and the gait speed is less than the significance level 0.05 (Yes in step S318), the feature amount is not affected by the gait speed, and therefore the determination system 1 sets the feature amount as a gait feature amount (step S319). On the other hand, if the significance probability p₂ of presence or absence of a correlation between the feature amount and the gait speed is equal to or more than the significance level 0.05 (No in step S318), the feature amount is affected by the gait speed, and therefore the process proceeds to A of FIG. 34.

If Yes in step S318 of FIG. 33, the determination system 1 obtains in FIG. 34 the regression line between the feature amount and the gait speed (step S320).

Next, the determination system 1 obtains the distance between the regression line of the gait speed and the feature amount (step S321).

Next, the determination system 1 divides the distance between the regression line of the gait speed and the feature amount into two groups (first set and second set) in terms of the presence and absence of hallux valgus, and calculates a significance probability p₃ of presence and absence of the difference between them (step S322).

If the significance probability p₃ of presence and absence of the difference in the distance between the regression line of the gait speed and the feature amount is significant is less than the significance level 0.05 (Yes in step S323), there is a significant difference, and therefore the determination system 1 sets the feature amount as a gait feature amount (step S324). On the other hand, if the significance probability p₃ of presence and absence of the difference in the distance between the regression line of the gait speed and the feature amount is significant is equal to or more than the significance level 0.05 (No in step S323), there is not a significant difference, and therefore the determination system 1 does not set the feature amount as a gait feature amount (step S325).

The above is the explanation on the selection method of the gait feature amount. Note that the processing along the flowcharts of FIGS. 33 and 34 may be performed by machine learning. For example, the determination system 1 is only required to be provided with a machine learning function, and is only required to select by machine learning the feature amount of the feature site extracted from the gait waveform data.

As described above, the determination system of the present example embodiment includes the data acquisition device and the anomaly detection device. The data acquisition device is installed in the footwear, measures a space acceleration and a space angular velocity, generates sensor data based on the measured space acceleration and space angular velocity, and transmits the generated sensor data to the anomaly detection device. The anomaly detection device includes the extraction unit and the detection unit. The extraction unit acquires sensor data from the sensor installed in the footwear, and uses the sensor data to extract a gait feature amount characteristic in gait of the pedestrian wearing the footwear. The detection unit detects an anomaly in the foot of the pedestrian walking wearing the footwear based on the gait feature amount extracted by the extraction unit.

According to the present example embodiment, sensor data is acquired from the sensor installed in the footwear, a gait feature amount characteristic in gait wearing the footwear is extracted using the sensor data, and an anomaly in the foot can be detected based on the extracted gait feature amount.

In one aspect of the present example embodiment, the detection unit determines the progression state of hallux valgus of the foot of the pedestrian wearing the footwear based on the gait feature amount extracted by the extraction unit. According to the present aspect, it is possible to determine the progression state of hallux valgus of the foot of the pedestrian based on the extracted gait feature amount.

For example, the detection unit estimates the progression state of hallux valgus using a model in which machine learning has been performed by using training data where the progression state of hallux valgus is used as a label and the gait feature amount characteristic in gait wearing the footwear is used as input data and the gait feature amount extracted by the extraction unit. According to this example, by inputting a gait feature amount to a model generated by machine learning, it is possible to estimate the progression state of hallux valgus according to the gait feature amount.

In one aspect of the present example embodiment, the detection unit estimates the angle formed by the center line of the first metatarsal bone and the center line of the first proximal phalanx of the foot of the pedestrian wearing the footwear based on the gait feature amount extracted by the extraction unit. According to the present aspect, it is possible to estimate the angle formed by the center line of the first metatarsal bone and the center line of the first proximal phalanx of the foot of the pedestrian based on the extracted gait feature amount.

For example, the detection unit estimates the HV angle by using a model in which machine learning is performed using training data where the HV angle formed by the center line of the first metatarsal bone and the center line of the first proximal phalanx is used as a label and the gait feature amount characteristic in gait wearing the footwear is used as input data, and the gait feature amount extracted by the extraction unit. According to this example, by inputting a gait feature amount to a model generated by machine learning, it is possible to estimate the HV angle according to the gait feature amount.

In one aspect of the present example embodiment, the extraction unit extracts a gait feature amount included in the gait waveform data obtained from the time series data of the sensor data acquired by gait of the pedestrian walking wearing the footwear. For example, the extraction unit extracts a gait feature amount included in a waveform of at least any of the mid-swing period and the initial stance period among the gait waveform data obtained from the time series data of the angular velocity about the axis of the lateral direction of the pedestrian. For example, the extraction unit extracts a gait feature amount included in a waveform of at least any of the mid-swing period and the initial stance period among the gait waveform data obtained from the time series data of the acceleration in the gravity direction. For example, the extraction unit extracts a gait feature amount included in a waveform of at least any of the initial swing period and the initial stance period among the gait waveform data obtained from the time series data of the acceleration in the traveling direction of the pedestrian. In the present aspect, the gait feature amount included in the gait waveform data is extracted. Therefore, according to the present aspect, an anomaly in the foot can be more accurately estimated using the characteristic gait feature amount extracted from the gait waveform data.

The timing of heel rocker in which the gait cycle included in the initial stance period is about 73% includes a period in which the acceleration in the gravity direction (Z direction) is converted into the traveling direction (Y direction) by rotation along the outer periphery of the heel coming into contact with the ground after heel contact. Therefore, it is estimated that the acceleration (FIG. 20) in the gravity direction (Z direction) rapidly decreases, and the acceleration (FIG. 24) in the traveling direction (Y direction) exhibits the maximum. A person susceptible to hallux valgus tends to be insufficient in arch formation of the arch of foot, is thus likely to have a flat-footed, pitter-patter gait, and tends to be fast in the angular velocity in heel rocker. Therefore, it is inferred that if gait is continued wearing footwear small relative to the size of the foot, a force continues to be applied to a direction where the thumb turns to be valgus, and thus the symptom of hallux valgus easily progresses.

In one aspect of the present example embodiment, the detection unit outputs distribution information relevant to the progression state of an anomaly in the foot of the pedestrian walking wearing the footwear. According to the present aspect, the pedestrian can acquire, in real time, distribution information relevant to the progression state of an anomaly in the foot.

### Second Example Embodiment

Next, an anomaly detection device according to the second example embodiment will be described with reference to the drawings.

The anomaly detection device of the present example embodiment is associated to the anomaly detection device 12 included in the determination system 1 of the first example embodiment. The anomaly detection device of the present example embodiment determines the presence or absence of an anomaly in a foot of a pedestrian using sensor data acquired by a sensor installed in footwear.

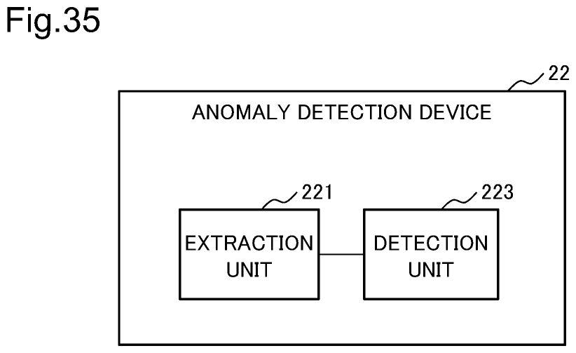

FIG. 35 is a block diagram illustrating an example of the configuration of an anomaly detection device 22 of the present example embodiment. The anomaly detection device 22 includes an extraction unit 221 and a detection unit 223.

The extraction unit 221 acquires sensor data from the sensor installed in the footwear. The extraction unit 221 uses the sensor data to extract a gait feature amount characteristic in gait of the pedestrian wearing the footwear.

The detection unit 223 detects an anomaly in the foot of the pedestrian walking wearing the footwear based on the gait feature amount extracted by the extraction unit 221.

According to the present example embodiment, it is possible to detect an anomaly in a foot based on features of gait of a pedestrian.

(Hardware)

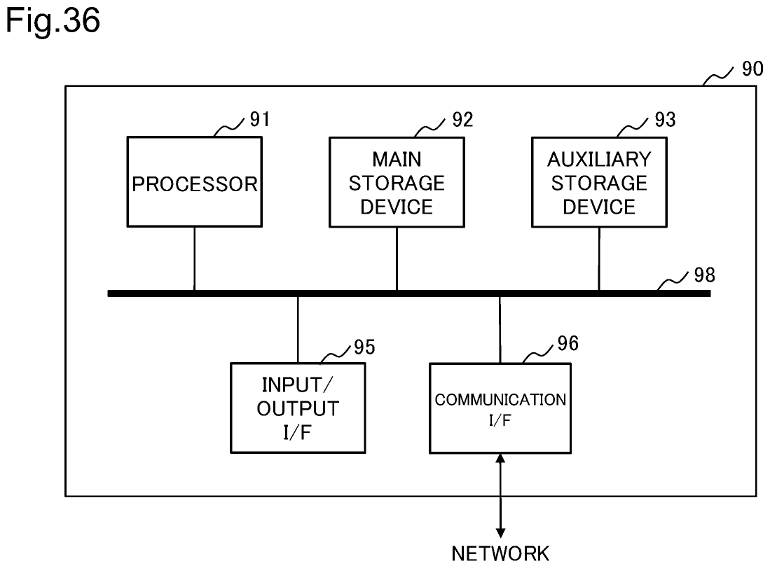

Here, the hardware configuration for executing the processing of the anomaly detection device according to each example embodiment will be described with an information processing device 90 of FIG. 36 as an example. Note that the information processing device 90 of FIG. 36 is a configuration example for executing the processing of the anomaly detection device of each example embodiment, and does not limit the scope of the present invention.

As in FIG. 36, the information processing device 90 includes a processor 91, a main storage device 92, an auxiliary storage device 93, an input/output interface 95, and a communication interface 96. In FIG. 36, the interface is abbreviated as I/F. The processor 91, the main storage device 92, the auxiliary storage device 93, the input/output interface 95, and the communication interface 96 are connected to one another via a bus 98 to be capable of data communication. The processor 91, the main storage device 92, the auxiliary storage device 93, and the input/output interface 95 are connected to a network such as the Internet or an intranet via the communication interface 96.

The processor 91 develops a program stored in the auxiliary storage device 93 or the like into the main storage device 92 and executes the developed program. In the present example embodiment, a configuration of using a software program installed in the information processing device 90 is sufficient. The processor 91 executes processing by the anomaly detection device according to the present example embodiment.

The main storage device 92 has a region in which a program is developed. The main storage device 92 is only required to be a volatile memory such as a dynamic random access memory (DRAM). A nonvolatile memory such as a magnetoresistive random access memory (MRAM) may be configured as and added to the main storage device 92.

The auxiliary storage device 93 stores various data. The auxiliary storage device 93 includes a local disk such as a hard disk or a flash memory. Various data can be stored in the main storage device 92, and the auxiliary storage device 93 can be omitted.

The input/output interface 95 is an interface for connecting the information processing device 90 and peripheral equipment. The communication interface 96 is an interface for connecting to an external system or device through a network such as the Internet or an intranet based on a standard or specifications. The input/output interface 95 and the communication interface 96 may be shared as an interface connected to external equipment.

The information processing device 90 may be connected with input equipment such as a keyboard, a mouse, and a touch screen as necessary. Those pieces of input equipment are used to input information and settings. In a case of using a touch screen as input equipment, the display screen of display equipment is only required to serve also as an interface of the input equipment. Data communication between the processor 91 and the input equipment may be mediated by the input/output interface 95.

Furthermore, the information processing device 90 may include display equipment for displaying information. In a case of including display equipment, the information processing device 90 desirably includes a display control device (not illustrated) for controlling display of the display equipment. The display equipment may be connected to the information processing device 90 via the input/output interface 95.

The above is an example of the hardware configuration for enabling the anomaly detection device according to each example embodiment of the present invention. Note that the hardware configuration of FIG. 36 is an example of a hardware configuration for executing the arithmetic processing of the anomaly detection device according to each example embodiment, and does not limit the scope of the present invention. A program that causes a computer to execute processing regarding the anomaly detection device according to each example embodiment is also included in the scope of the present invention.

Furthermore, a non-transitory recording medium (also referred to as program recording medium) that records a program according to each example embodiment is also included in the scope of the present invention. For example, the recording medium can be implemented by an optical recording medium such as a compact disc (CD) or a digital versatile disc (DVD). Furthermore, the recording medium may be implemented by a semiconductor recording medium such as a universal serial bus (USB) memory or a secure digital (SD) card, a magnetic recording medium such as a flexible disk, or another recording medium.

Components of the anomaly detection device of each example embodiment can be discretionarily combined. The components of the anomaly detection device of each example embodiment may be implemented by software or may be implemented by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

### REFERENCE SIGNS LIST

1 determination system
11 data acquisition device
12, 22 anomaly detection device
111 acceleration sensor
112 angular velocity sensor
113 signal processing unit
115 data transmission unit
120A first model
120B second model
121, 221 extraction unit
123, 223 detection unit

1. An anomaly detection device comprising:
a memory storing instructions; and
a processor connected to the memory and configured to execute the instructions to:
acquire sensor data including space acceleration and space angular velocity from a sensor installed in footwear worn by a pedestrian;
generate time series data of the space acceleration and the space angular velocity;
generate gait waveform data that is waveform data for one gait cycle by using the time series data of the space acceleration and the space angular velocity;
extract, from the gait waveform data, the spatial acceleration and the spatial angular velocity of a future site where an abnormal of a foot appears as a gait feature amount;
determine a progression state of hallux valgus of the foot of the pedestrian wearing the footwear based on the gait feature amount having been extracted; and
display content related to the progression state of hallux valgus of the pedestrian on a screen of a mobile terminal used by the pedestrian.

2. The anomaly detection device according to claim 1, wherein
the processor is configured to execute the instructions to
display a moving image including an ideal gait according to the progression state of hallux valgus of the pedestrian on the screen of the mobile terminal of the pedestrian.

3. The anomaly detection device according to claim 1, wherein
the processor is configured to execute the instructions to
display advice regarding a gait according to the progression state of hallux valgus of the pedestrian on the screen of the mobile terminal of the pedestrian.

4. The anomaly detection device according to claim 1, wherein
the processor is configured to execute the instructions to
display advice regarding a posture according to the progression state of hallux valgus of the pedestrian on the screen of the mobile terminal of the pedestrian.

5. The anomaly detection device according to claim 2, wherein
the processor is configured to execute the instructions to
estimate a progression state of the hallux valgus by using a model in which machine learning has been performed using training data where a progression state of the hallux valgus is used as a label and the gait feature amount characteristic in gait wearing the footwear is used as input data, and the gait feature amount having been extracted.

6. The anomaly detection device according to claim 2, wherein
the processor is configured to execute the instructions to
estimate an angle formed by a center line of the first metatarsal bone and a center line of the first proximal phalanx by using a model in which machine learning is performed using training data where an angle formed by a center line of the first metatarsal bone and a center line of the first proximal phalanx is used as a label and the gait feature amount characteristic in gait wearing the footwear is used as input data, and the gait feature amount having been extracted.

7. The anomaly detection device according to claim 1, wherein
the processor is configured to execute the instructions to
transmit the content related to the gait of the pedestrian optimized for healthcare use to the mobile terminal used by the pedestrian.

8. A determination system comprising:
the anomaly detection device according to claim 1; and
a data acquisition device that is installed in a footwear of a pedestrian, and configured to measure a space acceleration and a space angular velocity, generate the sensor data based on the space acceleration and the space angular velocity having been measured, and transmit the sensor data having been generated to the anomaly detection device.

9. An anomaly detection method executed by a computer, the method comprising:
acquiring sensor data including space acceleration and space angular velocity from a sensor installed in footwear worn by a pedestrian;
generate time series data of the space acceleration and the space angular velocity;
generating gait waveform data that is waveform data for one gait cycle by using the time series data of the space acceleration and the space angular velocity;
extracting, from the gait waveform data, the spatial acceleration and the spatial angular velocity of a future site where an anormal of a foot appears as a gait feature amount;
determining a progression state of hallux valgus of the foot of the pedestrian wearing the footwear based on the gait feature amount having been extracted; and
displaying content related to the progression state of hallux valgus of the pedestrian on a screen of a mobile terminal used by the pedestrian.

10. A non-transitory program recording medium recorded with a program causing a computer to perform the following processes:
acquiring sensor data including space acceleration and space angular velocity from a sensor installed in footwear worn by a pedestrian;
generate time series data of the space acceleration and the space angular velocity;
generating gait waveform data that is waveform data for one gait cycle by using the time series data of the space acceleration and the space angular velocity;
extracting, from the gait waveform data, the spatial acceleration and the spatial angular velocity of a future site where an anormal of a foot appears as a gait feature amount;
determining a progression state of hallux valgus of the foot of the pedestrian wearing the footwear based on the gait feature amount having been extracted; and
displaying content related to the progression state of hallux valgus of the pedestrian on a screen of a mobile terminal used by the pedestrian.
